(19) United States
(12) Patent Application Publication
(10) Pub. No.: US 2005/0043300 A1
(43) Pub. Date: Feb. 24, 2005

(54) Piperazine derivatives

(75) Inventors: Donald Stuart Middleton, Sandwich (GB); Charles Eric Mowbray, Sandwich (GB); Peter Thomas Stephenson, Sandwich (GB); David Howard Williams, Sandwich (GB)

Correspondence Address: AGOURON PHARMACEUTICALS, INC., 10350 NORTH TORREY PINES ROAD, LA JOLLA, CA, 92037, US

(73) Assignee: PFIZER INC.

(21) Appl. No.: 10/910,984

(22) Filed: Aug. 3, 2004

Related U.S. Application Data
(60) Provisional application No. 60/504,520, filed on Sep. 19, 2003.
(60) Provisional application No. 60/515,458, filed on Oct. 29, 2003.
(60) Provisional application No. 60/563,127, filed on Apr. 16, 2004.

(30) Foreign Application Priority Data
Aug. 14, 2003 (GB) 0319149.1
Sep. 22, 2003 (GB) 0322153.8
Mar. 24, 2004 (GB) 0406656.9

(51) Int. Cl. A61K 31/541; A61K 31/5377; A61K 31/517; A61K 31/496; C07D 417/02; C07D 413/02; C07D 43/02

(52) U.S. Cl. 514/227.8; 514/252.14; 514/252.11; 514/252.02; 514/235.5; 514/253.11; 514/253.06; 514/253.05; 514/248; 514/252.17; 544/60; 544/112; 544/238; 544/284; 544/295; 544/360; 544/363

(57) ABSTRACT

This invention relates to a compound of formula (I) 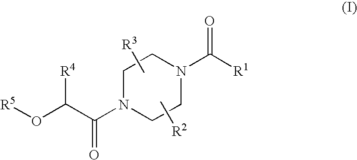
or pharmaceutically acceptable salts, solvates or derivatives thereof, wherein R¹ to R⁵ are defined in the description, and to processes for the preparation thereof, intermediates used in their preparation, compositions containing them and the uses of such derivatives. The compounds of the present invention inhibit the interaction of gp120 with CD4 and are therefore of use in the treatment of HIV, a retroviral infection genetically related to HIV, or AIDS.

This application claims the benefit of: United Kingdom Patent Application No. 0319149.1, filed on Aug. 14, 2003; United Kingdom Patent Application No. 0322153.8, filed on Sep. 22, 2003; United Kingdom Patent Application No. 0406656.9, filed on Mar. 24, 2004; U.S. Provisional Patent Application No. 60/504,520, filed on Sep. 19, 2003; U.S. Provisional Patent Application No. 60/515,458, filed on Oct. 29, 2003; and U.S. Provisional Patent Application No. 60/563,127, filed on Apr. 16, 2004, the contents of which are each incorporated by reference in their entirety.

### FIELD OF THE INVENTION

This invention relates to piperazine derivatives, to processes for their preparation, to compositions containing them and to their use.

More particularly, the present invention relates to the use of piperazine derivatives in the treatment of HIV, such as HIV-1, and genetically related retroviral infections (and the resulting acquired immune deficiency syndrome, AIDS).

Entry of HIV-1 into a target cell requires cell-surface CD4 and additional host cell cofactors. It is recognised that for efficient entry into target cells, human immunodeficiency viruses require a chemokine receptor, such as CCR5 or CXCR-4, as well as the primary receptor CD4. The principal cofactor for the entry mediated by the envelope glycoproteins of primary macrophage-trophic strains of HIV-1 is CCR5, a receptor for the β-chemokines RANTES, MIP-1α and MIP-1β (Deng et al., 1996, Nature, 38, 661-666). HIV attaches to the CD4 molecules on target cells through a region of its envelope protein, gp120. It is believed that the CD4 binding site on the gp120 of HIV interacts with the CD4 molecule on the target cell surface and undergoes conformational changes, which allow it to bind to further cell-surface receptors such as CCR5 or CXCR-4. This brings the viral envelope in closer proximity to the cell surface and allows interaction between gp41 on the viral envelope and a fusion domain on the host cell surface, subsequent fusion with the cell membrane and, ultimately, entry of the viral core into the cell. Accordingly, compounds that inhibit the binding of gp120 with CD4, and hence prevent the entry of HIV-1 into a target cell, should be useful in the treatment of HIV, such as HIV-1, and genetically related retroviral infections (and the resulting acquired immune deficiency syndrome, AIDS).

According to a first aspect of the present invention, there is provided a compound of formula (I) 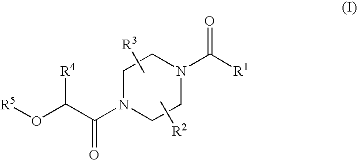
or a pharmaceutically acceptable salt, solvate or derivative thereof, wherein:
R¹ is phenyl or pyridyl, wherein said phenyl or pyridyl is optionally substituted by 1 or 2 atoms or groups selected from halo, C₁-C₆alkoxy, CF₃, OCF₃, or CN;
R² and R³ are independently H, or C₁-C₆alkyl;
R⁴ is C₁-C₆ alkyl;
R⁵ is phenyl; naphthyl; or a C-linked, 6 to 10 membered, mono- or bicyclic, aromatic or partially saturated, heterocycle wherein said heterocycle contains 1 to 4 nitrogen heteroatom(s), 1 or 2 nitrogen and 1 oxygen heteroatoms, or 1 or 2 nitrogen and 1 sulphur heteroatoms; wherein said phenyl, napthyl or heterocycle is optionally substituted by 1 to 3 atoms or groups selected from C₁-C₆ alkyl, C₁-C₆ fluoroalkyl, C₃-C₇ cycloalkyl, phenyl, OH, C₁-C₆ alkoxy, C₁-C₆ alkoxy C₁-C₆ alkyl, OC₁-C₆fluoroalkyl, C₀-C₂ alkylene NR⁶R⁷, halo, C₀-C₂ alkylene CN, C₀-C₂ alkylene CO₂R⁸, C₀-C₂ alkylene CONR⁶R⁷, C₀-C₂ alkylene SR⁹, C₀-C₂ alkylene SOR⁹, C₀-C₂ alkylene SO₂R⁹, C₀-C₂ alkylene SO₂NR⁶R⁷, C₀-C₂ alkylene NR⁸COR⁹, C₀-C₂ alkylene NR⁸CONR⁶R⁷, C₀-C₂ alkylene NR⁸SO₂R⁹, or C₀-C₂ alkylene R¹⁰, or, where R⁵ is a heterocycle, oxo;
R⁶ and R⁷ are independently H, C₁-C₆ alkyl, C₃-C₇ cycloalkyl, phenyl or R¹⁰; or when taken together with the nitrogen to which they are attached form an optionally substituted azetidine, pyrrolidine, piperidine, morpholine, or thiomorpholine ring; wherein the said substituents are 1 or 2 groups selected from C₁-C₆ alkyl, or C₀-C₆ alkylene NH₂;
R⁸ is H, C₁-C₆ alkyl or phenyl;
R⁹ is C₁-C₆ alkyl or phenyl; and
R¹⁰ is imidazolyl, pyrazolyl, triazolyl, thienyl, furyl, thiazolyl, oxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl, each optionally substituted by 1 to 3 atoms or groups selected from C₁-C₆ alkyl, C₁-C₆ alkoxy, cyano or halo.

The term “alkyl” as a group or part of a group includes straight chain and branched groups. Examples of alkyl include methyl, ethyl, n-propyl, i-propyl, n-butyl, i-butyl, sec-butyl and t-butyl. The term “C₃₋₇ cycloalkyl” means cyclopropyl, cyclobutyl, cyclopentyl, cyclohexyl or cycloheptyl. The term “halo” means fluoro, chloro, bromo or iodo.

In a further embodiment, R¹ is phenyl or pyridyl, wherein said phenyl or pyridyl is optionally substituted by 1 or 2 atoms or groups selected from halo.

In yet a further embodiment, R¹ is phenyl, fluorophenyl or pyridyl.

In yet a further embodiment, R¹ is phenyl.

In yet a further embodiment, R² is C₁-C₄alkyl.

In yet a further embodiment, R² is methyl.

In yet a further embodiment, R³ is H.

In yet a further embodiment, R⁴ is C₁-C₄alkyl.

In yet a further embodiment, R⁴ is methyl.

In yet a further embodiment, R⁵ is an optionally substituted phenyl, naphthyl, pyridyl, indazolyl, benzimidazolyl, quinolinyl, isoquinolinyl, quinazolinyl, benzopiperidinyl or benzoxazolyl; wherein said substituents are 1 to 3 atoms or groups selected from C₁-C₆ alkyl, C₁-C₆ alkoxy, halo, CN, CO₂R⁸, CONR⁶R⁷, or R¹⁰.

In yet a further embodiment, R⁵ is an optionally substituted phenyl or pyridyl, wherein said substituents are 1 to 3 groups selected from C₁-C₆ alkoxy, CO₂R⁸, or CONR⁶R⁷.

In yet a further embodiment, R⁶ is H or C₁-C₄ alkyl.

In yet a further embodiment, R⁷ is H, C₁-C₄ alkyl or C₃-C₆ cycloalkyl.

In yet a further embodiment, R⁸ is C₁-C₄ alkyl.

In yet a further embodiment, R¹⁰ is imidazolyl, pyrazolyl, triazolyl or oxadiazolyl, each optionally substituted by 1 to 3 atoms or groups selected from C₁-C₄ alkyl, C₁-C₄ alkoxy, cyano or halo.

In yet a further embodiment, there is provided a compound of formula (Ia)

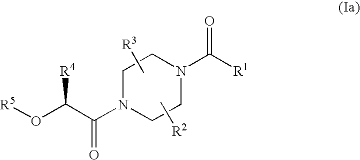

or a pharmaceutically acceptable salt, solvate or derivative thereof, wherein R¹, R², R³, R⁴ and R⁵ are as defined hereinabove with respect to a compound of formula (I), including all combinations of particular described embodiments thereof.

In yet a further embodiment, there is provided a compound of formula (Ib)

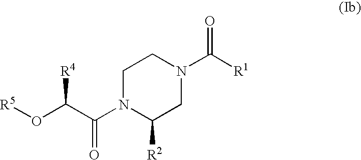

or a pharmaceutically acceptable salt, solvate or derivative thereof, wherein R¹, R², R⁴ and R⁵ are as defined hereinabove with respect to a compound of formula (I), including all combinations of particular described embodiments thereof.

It is to be understood that the invention covers all combinations of particular embodiments of the invention as described hereinabove, consistent with the definition of compounds of formula (I).

The compounds of the invention include compounds of formula (I) and pharmaceutically acceptable salts, solvates or derivatives thereof (wherein derivatives include complexes, polymorphs, prodrugs and isotopically-labeled compounds, as well as salts, solvates and salt solvates thereof), and isomers thereof. In a further embodiment, the compounds of the invention are the compounds of formula (I) and pharmaceutically acceptable salts and solvates thereof, in particular the compounds of formula (I). It is to be understood that the aforementioned compounds of the invention include polymorphs and isomers thereof.

Pharmaceutically acceptable salts of the compounds of formula (I) include the acid addition and base salts thereof.

Suitable acid addition salts are formed from acids which form non-toxic salts. Examples include the acetate, aspartate, benzoate, besylate, bicarbonate, bisulphate, borate, bromide, camsylate, carbonate, chloride, citrate, edisylate, esylate, formate, fumarate, gluceptate, gluconate, glucuronate, hexafluorophosphate, hibenzate, hydrobromide, hydrochloride, hydroiodide, iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, sulphate, tartrate, tosylate and trifluoroacetate salts.

Suitable base salts are formed from bases which form non-toxic salts. Examples include the aluminium, arginine, benzathine, calcium, choline, diethylamine, diolamine, lycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The salt may precipitate from solution and be collected by filtration or may be recovered by evaporation of the solvent. The degree of ionisation in the salt may vary from completely ionised to almost non-ionised.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

Complexes include clathrates, i.e. drug-host inclusion complexes wherein, in contrast to the aforementioned solvates, the drug and host are present in stoichiometric or non-stoichiomnetric amounts. Also included are complexes of the pharmaceutical drug which contain two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the present invention may have the ability to crystallize in more than one form, a characteristic known as polymorphism, and all such polymorphic forms (“polymorphs”) are encompassed within the scope of the invention. Polymorphism generally can occur as a response to changes in temperature or pressure or both, and can also result from variations in the crystallization process. Polymorphs can be distinguished by various physical characteristics, and typically the x-ray diffraction patterns, solubility behavior, and melting point of the compound are used to distinguish polymorphs.

Certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985).

Some examples of prodrugs in accordance with the invention include:
(i) where the compound of formula (I) contains a carboxylic acid functionality (—COOH), an ester thereof, for example, replacement of the hydrogen with (C₁-C₆)alkyl;
(ii) where the compound of formula (I) contains an alcohol functionality (—OH), an ether thereof, for example, replacement of the hydrogen with (C₁-C₆)alkanoyloxymethyl; and
(iii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types in accordance with the invention may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include:
(i) where the compound of formula (I) contains a methyl group, an hydroxymethyl derivative thereof (—CH₃->—CH₂OH):
(ii) where the compound of formula (I) contains an alkoxy group, an hydroxy derivative thereof (—OR->—OH);
(iii) where the compound of formula (I) contains a tertiary amino group, a secondary amino derivative thereof (—NR¹R²->—NHR¹ or —NHR²);
(iv) where the compound of formula (I) contains a secondary amino group, a primary derivative thereof (—NHR¹->—NH₂);
(v) where the compound of formula (I) contains a phenyl moiety, a phenol derivative thereof (-Ph->-PhOH); and
vi) where the compound of formula (I) contains an amide group, a carboxylic acid derivative thereof (—CONH₂->COOH).

In view of the definition of R⁴, compounds of formula (I) contain one or more asymmetric carbon atoms and therefore exist as two or more optical isomers. Where a compound of formula (I) contains an alkenyl or alkenylene group, geometric cisltrans (or Z/E) isomers are possible, and where the compound contains, for example, a keto or oxime group or an aromatic moiety, tautomeric isomerism (‘tautomerism’) may occur. It follows that a single compound may exhibit more than one type of isomerism.

Included within the scope of the present invention are all optical isomers, geometric isomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cisltrans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, N.Y., 1994).

The present invention also includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, ie. ¹⁴C, are particularly useful for this purpose in view of their ease of incorporation and ready means of detection.

Substitution with heavier isotopes such as deuterium, ie. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Preferred compounds of formula (I) include the compounds of Examples 1-94; and pharmaceutically acceptable salts, solvates or derivatives thereof. Particularly preferred compounds of formula (I) include:
(2S)-1-[(2R)-4-Benzoyl-2-methyl-piperazin-1-yl)]-2-(3-methyl-1H-indazol-4-yloxy)-propan-1-one;
(2S)-1-[(2R)-4-Benzoyl-2-methyl-piperazin-1-yl]-2-[2-(2H-pyrazol-3-ylamino)-quinolin-5-yloxy]-propan-1-one;
5-{(1S)-2-[(2R)-4-Benzoyl-2-methyl-piperazin-1-yl]-1-methyl-2-oxo-ethoxy}-isoquinoline-1-carboxylic acid methylamide;
(2S)-1-[(2R)-4-Benzoyl-2-methyl-piperazin-1-yl]-2-(8-chloro-2-methylamino-quinolin-5-yloxy)-propan-1-one;
(2S)-1-[(2R)-4-Benzoyl-2-methyl-piperazin-1-yl]-2-[1-(2H-pyrazol-3-ylamino)-isoquinolin-5-yloxy]-propan-1-one;
4-{(1S)-2-[(2R)-4-Benzoyl-2-methyl-piperazin-1-yl]-1-methyl-2-oxo-ethoxy}-3-methoxy-N-methyl-benzamide;
5-{(1S)-2-[(2R)-4-Benzoyl-2-methyl-piperazin-1-yl]-1-methyl-2-oxo-ethoxy}-4-methoxy-pyridine-2-carboxylic acid methylamide;
5-{(1S)-2-[(2R)-4-Benzoyl-2-methyl-piperazin-1-yl]-1-methyl-2-oxo-ethoxy}-4-methoxy-pyridine-2-carboxylic acid amide;
5-{(1S)-2-[(2R)-4-Benzoyl-2-methyl-piperazin-1-yl]-1-methyl-2-oxo-ethoxy}-4-methoxy-pyridine-2-carboxylic acid ethylamide;
5-{(1S)-2-[(2R)-4-Benzoyl-2-methyl-piperazin-1-yl]-1-methyl-2-oxo-ethoxy}-4-methoxy-pyridine-2-carboxylic acid cyclopropylamide;
and pharmaceutically acceptable salts, solvates or derivatives thereof.

In the general processes, and schemes, that follow: R¹ to R⁹ are as previously defined unless otherwise stated; X is halo or hydroxy; Y is a leaving group, such as chloro, bromo, tosylate, mesylate or hydroxy; DMF is N,N-dimethylformamide; DMSO is dimethylsulphoxide; THF is tetrahydrofuran; WSCDI is 1-(3-dimethylaminopropyl)-3-ethylcarbodiimide hydrochloride; DCC is N,N′-dicyclohexylcarbodiimide; HOAT is 1-hydroxy-7-azabenzotriazole; HOBt is 1-hydroxybenzotriazole hydrate; HBTU is O-(1H-benzotriazol-1-yl)-N,N,N′,N′-tetramethyluronium hexafluorophosphate; PyBOP® is benzotriazol-1-yloxytris(pyrrolidino)phosphonium hexafluorophosphate; PyBrOP is bromo-tris-pyrrolidino-phosphonium hexafluorophosphate; Hunigs base is N-ethyldiisopropylamine; and Mukaiyama's reagent is 2-chloro-1-methylpyridinium iodide.

Compounds of formula (I) may be prepared by any methods known for the preparation of compounds of analogous structure.

Compounds of formula (I), and intermediates thereto, may be prepared according to the schemes that follow.

It will be appreciated by those skilled in the art that certain of the procedures described in the schemes for the preparation of compounds of formula (I) or intermediates thereto may not be applicable to some of the possible substituents.

It will be further appreciated by those skilled in the art that it may be necessary or desirable to carry out the transformations described in the schemes in a different order from that described, or to modify one or more of the transformations, to provide the desired compound of formula (I).

It will be still further appreciated by those skilled in the art that, as illustrated in the schemes that follow, it may be necessary or desirable at any stage in the synthesis of compounds of formula (I) to protect one or more sensitive groups in the molecule so as to prevent undesirable side reactions. In particular, it may be necessary or desirable to protect amino groups. The protecting groups used in the preparation of compounds of formula (I) may be used in conventional manner. See, for example, those described in ‘Protective Groups in Organic Synthesis’ by Theodora W Green and Peter G M Wuts, third edition, (John Wiley and Sons, 1999), in particular chapter 7, pages 494-653 (“Protection for the Amino Group”), incorporated herein by reference, which also describes methods for the removal of such groups.

The amino protecting groups boc, benzyloxycarbonyl, benzyl and acetyl are of particular use in the preparation of compounds of formula (I) and intermediates thereto.

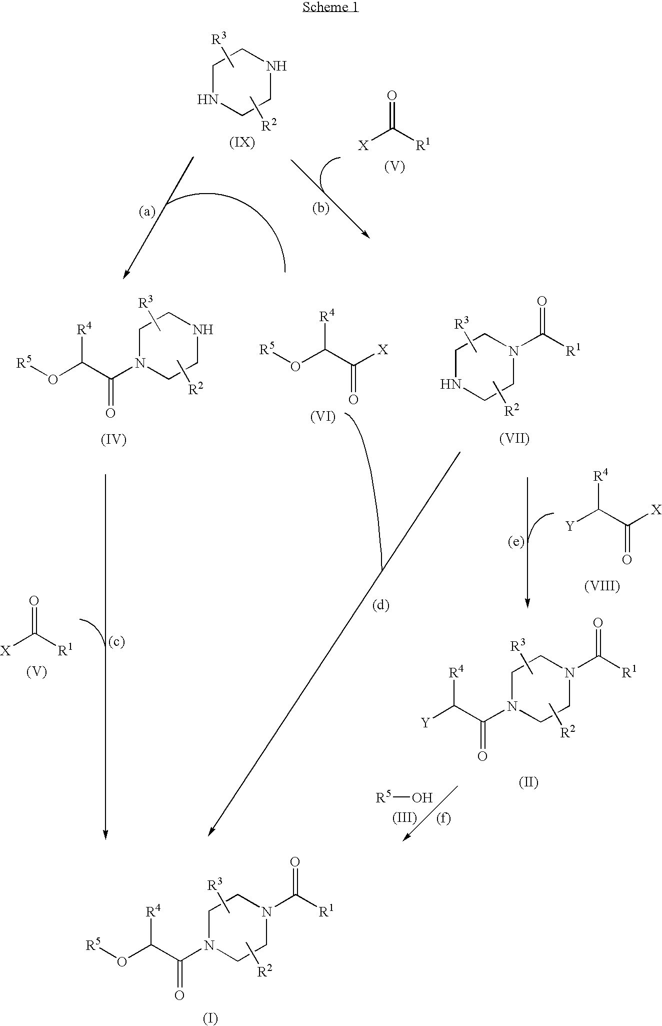

With specific reference to scheme 1, the transformations depicted therein may be effected as follows:

(a)-(e) Acid-amine Coupling Reactions

Typically the acid chloride or acid bromide of formulae (V), (VI) or (VIII), and the appropriate piperazine of formulae (IV), (VII) or (IX), optionally with an excess of an acid acceptor such as triethylamine or N-ethyl-N,N-diisopropylamine, are reacted in a solvent, such as an haloalkane (e.g. dichloromethane) or an ether (e.g. THF) at room temperature for 1-24 hours. The reactions may conveniently be carried out by reacting the relevant piperazine with 1.1 equivalents of the relevant acid chloride in dichloromethane at room temperature for 1 hour.

In a further embodiment the acid of formulae (V), (VI) or (VIII), activated by suitable reagents such as WSCDI/DCC and HOBt/HOAt, the appropriate piperazine of formulae (IV), (VII) or (IX), and an excess of an acid acceptor such as triethylamine or N-ethyl-N,N-diisopropylamine, are reacted in a solvent such as an haloalkane (e.g. dichloromethane), an ether (e.g. THF) or DMF at room temperature for 4-48 hours. The reactions may conveniently be carried out by reacting the relevant piperazine, 1.4 equivalents of WSCDI, 1.4 equivalents of HOBt, 2.2 equivalents of triethylamine and 1.1 equivalents of the relevant carboxylic acid in dichloromethane at room temperature for 18 hours.

In yet a further embodiment, the acid of formulae (V), (VI) or (VII), the appropriate piperazine of formulae (IV), (VII) or (IX), and either HBTU, PyBOP, PyBrOP or Mukaiyama's reagent, and an excess of an acid acceptor such as triethylamine or N-ethyl-N,N-diisopropylamine, may be reacted in a solvent such as an haloalkane (e.g. dichloromethane) or an ether (e.g. THF) at room temperature for 4-24 hours. The reactions may conveniently be carried out by reacting the relevant piperazine, 1.0 equivalents of the relevant carboxylic acid and 1.5 equivalents of HBTU in either dichloromethane or DMF at room temperature for 14 hours.

(f) Nucleophilic Substitution

i) When Y=Cl, Br, mesylate, tosylate:

Typically compounds of formulae (II) and (III), with an acid acceptor, such as triethylamine , N-ethyl-N,N-diisopropylamine or an alkali metal carbonate, are reacted in a solvent such as an haloalkane (e.g. dichloromethane), an ether (e.g. THF) or acetone at a temperature between room temperature and reflux for 1-24 hours. The reactions may conveniently be carried out by reacting compounds of formulae (II) and (III) with 1.0 equivalents of caesium carbonate in acetone at reflux for 14 hours.

ii) When Y=OH:

Typically compounds of formulae (II) and (III), triphenylphosphine or tri-o-tolylphosphine, and either diethyl azodicarboxylate or di-isopropyl azodicarboxylate, are reacted in a solvent such as an haloalkane (e.g. dichloromethane) or an ether (e.g. THF) at a temperature between room temperature and reflux for 1-24 hours. The reactions may conveniently be carried out by reacting compounds of formulae (II) and (III) with 1.2 equivalents of triphenylphosphine and 1.1 equivalents of di-isopropyl azodicarboxylate in THF at room temperature for 14 hours.

Compounds of formula (VI) in reaction steps (a) and (d) may be prepared according to scheme 1a that follows. 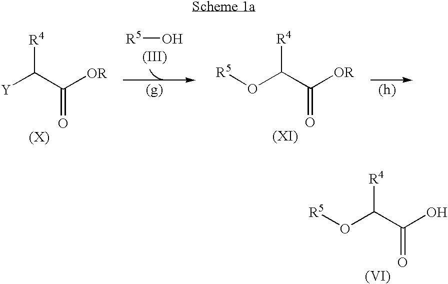
wherein R is a lower alkyl such as C₁-C₆ alkyl.
(g) Nucleophilic Substitution

Nucleophilic substitution may be effected according to the conditions described for step (f) hereinabove.

(h) Ester Hydrolysis

Typically, compounds of formula (XI), aqueous alkali metal hydroxide solution or aqueous hydrochloric acid solution, and an optional co-solvent such as ethanol or dioxane, are heated at a temperature between 60 and 100° C. for 1-18 hours. The reactions may conveniently be carried out by heating compounds of formula (XI) in aqueous 1N sodium hydroxide solution and dioxane heated at 60° C. for 2 hours.

Certain intermediates described above are novel compounds, and it is to be understood that all novel intermediates herein form further aspects of the present invention. Compounds of formulae (II), (IV) and (VII) are key intermediates and represent a particular aspect of the present invention.

Compounds of formulae (III), (V), (VIII), (IX) and (X) are either known compounds or may be prepared by conventional chemistry.

According to another aspect, the invention provides the following processes for preparing compounds of formula (I).

According to a first process (A), compounds of formula (I) may be prepared by coupling an acid of formula (V) 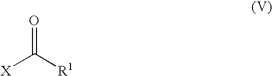
with a piperazine of formula (IV), under conventional acid-amine coupling conditions. 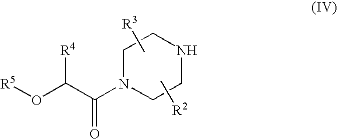
Conveniently, acid-amine coupling is effected under the conditions described hereinabove in connection with scheme 1, steps (a)-(e).

According to a second process (B), compounds of formula (I) may be prepared by coupling an acid of formula (VI) 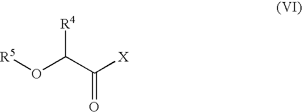
with a piperazine of formula (VII), under conventional acid-amine coupling conditions.

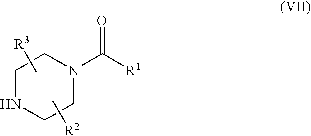

Conveniently, acid amine coupling is effected under the conditions described hereinabove in connection with scheme 1, steps (a)-(e).

According to a third process (C), compounds of formula (I) may be prepared by nucleophilic substitution of a compound of formula (II) 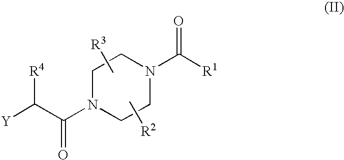
with an alcohol R⁵OH of formula (III) under conventional conditions. Conveniently, nucleophilic substitution is effected under the conditions described hereinabove in connection with scheme 1, step (f).

According to a fourth process (D), compounds of formula (I) may be prepared from other compounds of formula (I) by functional group interconversion under conventional conditions. For example, compounds of formula (I) which contain an ester group may be converted to corresponding compounds of formula (I) which contain a primary or secondary amide group by reacting the former with ammonia or a primary amine respectively.

The compounds of the invention inhibit the interaction of gp120 with CD4 and are therefore of use in the treatment of HIV, a retroviral infection genetically related to HIV, and AIDS.

Accordingly, in another aspect the invention provides a compound of formula (I) or a pharmaceutically acceptable salt, solvate or derivative thereof for use as a medicament.

In another aspect the invention provides a compound of formula (I) or a pharmaceutically acceptable salt, solvate or derivative thereof for use in the treatment of a HIV, a retroviral infection genetically related to HIV, or AIDS.

In another aspect the invention provides the use of a compound of formula (I) or of a pharmaceutically acceptable salt, solvate or derivative thereof for the manufacture of a medicament for the treatment of a HIV, a retroviral infection genetically related to HIV, or AIDS.

In another aspect the invention provides a method of treatment of a mammal suffering from HIV, a retroviral infection genetically related to HIV, or AIDS which comprises treating said mammal with an effective amount of a compound of formula (I) or a pharmaceutically acceptable salt, solvate or derivative thereof.

The compounds of the invention may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or in any combination thereof. Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavours, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, 15 Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et a/, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the compound. Examples of such formulations include drug-coated stents and PGLA microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Biojec™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound comprising, for example, ethanol (optionally, aqueous ethanol) or a suitable alternative agent for dispersing, solubilising, or extending release of the compound, the propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations-for inhaled/intranasal administration may be formulated to be immediate and/or modified release using, for example, poly(DL-1actic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of the invention. The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a compound of the invention in combination with another therapeutic agent, for example, for the purpose of treating a particular disease or condition, it is within the scope of the present invention that two or more pharmaceutical compositions, at least one of which contains a compound of the invention, may conveniently be combined in the form of a kit suitable for coadministration of the compositions.

Thus the kit of the invention comprises two or more separate pharmaceutical compositions, at least one of which contains a compound of formula (I) or a pharmaceutically acceptable salt, solvate or derivative thereof, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, having a weight of about 65 to 70 kg, the total daily dose of a compound of the invention is typically in the range 1 to 10000 mg, such as 10 to 1000 mg, for example 25 to 500 mg, depending, of course, on the mode of administration, the age, condition and weight of the patient, and will in any case be at the ultimate discretion of the physician. The total daily dose may be administered in single or divided doses.

Accordingly in another aspect the invention provides a pharmaceutical composition including a compound of formula (I) or a pharmaceutically acceptable salt, solvate or derivative thereof together with one or more pharmaceutically acceptable excipients, diluents or carriers.

The compounds of formula (I) and their pharmaceutically acceptable salts, solvates and derivatives have the advantage that they are more selective, have a more rapid onset of action, are more potent, are better absorbed, are more stable, are more resistant to metabolism, have a reduced ‘food effect’, have an improved safety profile or have other more desirable properties (e.g. with respect to solubility or hygroscopicity) than the compounds of the prior art.

The compounds of formula (I) and their pharmaceutically acceptable salts, solvates and derivatives may be administered alone or as part of a combination therapy. Thus included within the scope of the present invention are embodiments comprising coadministration of, and compositions which contain, in addition to a compound of the invention, one or more additional therapeutic agents. Such multiple drug regimens, often referred to as combination therapy, may be used in the treatment and prevention of infection by human immunodeficiency virus, HIV. The use of such combination therapy is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature. A recommended treatment for HIV is a combination drug treatment called Highly Active Anti-Retroviral Therapy, or HAART. HAART combines three or more HIV drugs. Thus, the methods of treatment and pharmaceutical compositions of the present invention may employ a compound of the invention in the form of monotherapy, but said methods and compositions may also be used in the form of combination therapy in which one or more compounds of the invention are coadministered in combination with one or more additional therapeutic agents such as those described in detail further herein.

In a further embodiment of the invention, combinations of the present invention include treatment with a compound of formula (I), or a pharmaceutically acceptable salt, solvate or derivative thereof, and one or more additional therapeutic agents selected from the following: HIV protease inhibitors, including but not limited to indinavir, ritonavir, saquinavir, nelfinavir, lopinavir, amprenavir, atazanavir, tipranavir, AG1859 and TMC 114; non-nucleoside reverse transcriptase inhibitors (NNRTIs), including but not limited to nevirapine, delavirdine, capravirine, efavirenz, GW-8248, GW-5634 and TMC125; nucleoside/nucleotide reverse transcriptase inhibitors, including but not limited to zidovudine, didanosine, zalcitabine, stavudine, lamivudine, abacavir, adefovir dipivoxil, tenofovir, emtricitabine and alovudine;
CCR5 antagonists, including but not limited to:
N-{(1S)-3-[3-(3-isopropyl-5-methyl-4H-1,2,4-triazol-4-yl)-exo-8-azabicyclo[3.2.1]oct-8-yl]-1-phenylpropyl}-4,4-difluorocyclohexanecarboxamide or a pharmaceutically acceptable salt, solvate or derivative thereof,
methyl 1-endo{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or a pharmaceutically acceptable salt, solvate or derivative thereof,
methyl 3-endo-{8-[(3S)-3-(acetylamino)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate or a pharmaceutically acceptable salt, solvate or derivative thereof,
ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or a pharmaceutically acceptable salt, solvate or derivative thereof,
Sch-D, ONO-4128, GW-873140, AMD-887 and CMPD-167; other agents which inhibit the interaction of gp120 with CD4, including but not limited to BMS806 and BMS-488043; other agents which inhibit the entry of HIV into a target cell, including but not limited to enfuviritide, T1249, PRO 542 and PRO 140; integrase inhibitors, including but not limited to L-870,810; and RNaseH inhibitors.

There is also included within the scope the present invention, combinations of a compound of formula (I), or a pharmaceutically acceptable salt, solvate or derivative thereof, together with one or more additional therapeutic agents independently selected from the group consisting of proliferation inhibitors, e.g. hydroxyurea; immunomodulators, such as granulocyte macrophage colony stimulating growth factors (e.g. sargramostim), and various forms of interferon or interferon derivatives; other chemokine receptor agonists/antagonists, such as CXCR4 antagonists (e.g. AMD-070); tachykinin receptor modulators (e.g. NK1 antagonists) and various forms of interferon or interferon derivatives; agents which substantially inhibit, disrupt or decrease viral transcription or RNA replication such as inhibitors of tat (transcriptional trans activator) or nef (negative regulatory factor); agents which substantially inhibit, disrupt or decrease translation of one or more proteins expressed by the virus (including, but not limited to, down regulation of protein expression or antagonism of one or more proteins) other than reverse transcriptase, such as Tat or Nef; agents which influence, in particular down regulate, CCR5 receptor expression; chemokines that induce CCR5 receptor internalisation such MIP-1α, MIP-1β, RANTES and derivatives thereof; and other agents that inhibit viral infection or improve the condition or outcome of HIV-infected individuals through different mechanisms.

Agents which influence (in particular down regulate) CCR5 receptor expression include immunosupressants, such as calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-{(3R,4R)-4-methyl-3-[methyl-(7H-pyrrolo[2,3-d]pyrimidin-4-yl)-amino]-piperidin-1-yl}-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab); and agents which interfere with cell activation or cell cycling, such as rapamycin.

There is also included within the scope the present invention, combinations of a compound of formula (I), or a pharmaceutically acceptable salt, solvate or derivative thereof, together with one or more additional therapeutic agents which slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C₉, CYP2C19 and CYP3A4. Suitable agents that may be used to inhibit CYP 3A4 include, but are not limited to, ritonavir, saquinavir or ketoconazole.

It will be appreciated by a person skilled in the art, that a combination drug treatment, as described herein above, may comprise two or more compounds having the same, or different, mechanism of action. Thus, by way of illustration only, a combination may comprise a compound of the invention and: one or more NNRTIs; one or more NRTIs and a PI; one or more NRTIs and a CCR5 antagonist; a PI; a PI and an NNRTI; and so on.

In addition to the requirement of therapeutic efficacy, which may necessitate the use of therapeutic agents in addition to the compounds of the invention, there may be additional rationales which compel or highly recommend the use of a combination of a compound of the invention and another therapeutic agent, such as in the treatment of diseases or conditions which directly result from or indirectly accompany the basic or underlying disease or condition. For example, it may be necessary or at least desirable to treat Hepatitis C Virus (HCV), Hepatitis B Virus (HBV), Human Papillomavirus (HPV), opportunistic infections (including bacterial and fungal infections), neoplasms, and other conditions which occur as the result of the immune-compromised state of the patient being treated. Other therapeutic agents may be used with the compounds of the invention, e.g., in order to provide immune stimulation or to treat pain and inflammation which accompany the initial and fundamental HIV infection.

Accordingly, therapeutic agents for use in combination with the compounds of formula (I) and their pharmaceutically acceptable salts, solvates and derivatives also include: interferons, pegylated interferons (e.g. peginterferon alfa-2a and peginterferon alfa-2b), lamivudine, ribavirin, and emtricitabine for the treatment of hepatitis; antifungals such as fluconazole, itraconazole, and voriconazole; antibacterials such as azithromycin and clarithromycin; interferons, daunorubicin, doxorubicin, and paclitaxel for the treatment of AIDS related Kaposi's sarcoma; and cidofovir, fomivirsen, foscarnet, ganciclovir and valcyte for the treatment of cytomegalovirus (CMV) retinitis.

Further combinations for use according to the invention include combination of a compound of formula (I), or a pharmaceutically acceptable salt, solvate or derivative thereof with a CCR1 antagonist, such as BX-471; a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab), a HMG CoA reductase inhibitor, such as a statin (e.g. atorvastatin); or an immunosuppressant, such as cyclosporin or a macrolide such as tacrolimus.

In the above-described combinations, the compound of formula (I) or a pharmaceutically acceptable salt, solvate or derivative thereof and other therapeutic agent(s) may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Accordingly, in a further aspect the invention provides a pharmaceutical composition comprising a compound of formula (I) or a pharmaceutically acceptable salt, solvate or derivative thereof and one or more additional therapeutic agents.

It is to be appreciated that all references herein to treatment include curative, palliative and prophylactic treatment.

The invention is illustrated by the following Examples and Preparations in which the following further abbreviations may be used:
0.88 ammonia=concentrated ammonium hydroxide solution, 0.88 SG
h=hour
min=minute
LRMS=low resolution mass spectrum
APCI+=atmospheric pressure chemical ionisation
ESI+=electrospray ionisation
NMR=nuclear magnetic resonance
tlc—thin layer chromatography
Me=methyl

### EXAMPLE 1

### (2S)-1-[(2R)-4-Benzoyl-2-methyl-piperazin-1-yl]-2-(quinolin-5-yloxy)-prooan-1-one

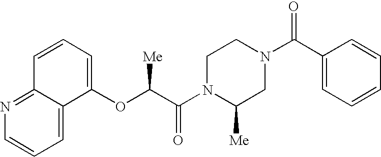 A solution of (2S)-2-(quinolin-5-yloxy)-propionic acid sodium salt (Preparation 2) (1.0 g, 4.6 mmol), (3R)-(3-methyl-piperazin-1-yl)-phenyl-methanone (J. Med. Chem. (2000), 43(23), 4499) (0.94 g, 4.6 mmol), O-(1H-benzotriazol-1-yl)-N,N,N′,N′-tetramethyluronium hexafluorophosphate (2.62 g, 6.9 mmol) and triethylamine (1.93 ml, 13.8 mmol) in N,N-dimethylformamide (30 ml) was stirred under a nitrogen atmosphere at room temperature for 14 hours. The reaction mixture was diluted with dichloromethane (100 ml), washed with water (3×50 ml), dried (MgSO₄) and solvent evaporated under reduced pressure. The crude product was purified by column chromatography on silica gel eluting with dichloromethane: methanol (98:2) to afford the title compound as a white solid (0.91 g).

¹H NMR (400 MHz, CDCl₃): δ: 8.90 (1H, m), 8.60 (1H, m), 7.70 (1H, m), 7.55 (1H, m), 7.55 (1H, m) 7.20-7.40 (6H, m), 6.85 (1H, m), 5.15 (1H, m), 4.80-2.80 (7H, m), 1.70 (3H, d), 0.90 (3H, br s) ppm. LRMS (APCI+): m/z [M+H]⁺ 404.

### EXAMPLE 2

### 3-[2-((2R)-4-Benzoyl-2-methyl-piperazin-1-yl)-1-methyl-2-oxo-ethoxy]-2-methyl-benzoic acid methyl ester

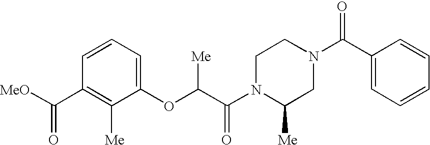 Cesium carbonate (0.39 g, 1.2 mmol) was added portionwise to a solution of 1-[(2R)-4-benzoyl-2-methyl-piperazin-1-yl]-2-bromo-propan-1-one (Preparation 3) (0.41 g, 1.2 mmol) and 3-hydroxy-2-methyl-benzoic acid methyl ester (Tet Lett. (2000), 41(11), 1741) (0.2 g, 1.2 mmol) in acetone (10 ml) and the mixture heated under reflux for 14 hours. The cooled reaction mixture was evaporated to dryness under reduced pressure and the residue partitioned between dichloromethane (50 ml) and water (10 ml). The organic phase was separated and the aqueous phase extracted with dichloromethane (3×30 ml), the combined organic extracts were dried (MgSO₄) and solvent evaporated under reduced pressure to afford the title compound as a white solid (0.5 g).

¹H NMR (400 MHz, CDCl₃): δ: 7.50-7.30 (6H, m), 7.15 (1H, m), 6.95 (1H, m), 4.95 (1H, m), 4.80-2.80 (7H, m), 3.90 (3H, s), 2.40 (3H, s), 1.65 (3H, d), 1.20 (3H, br s) ppm. LRMS (APCI+): m/z [M+H]⁺ 425.

Examples 3-12 were prepared by the method described above for Example 1, using the corresponding acid of formula (VI) and piperazine of formula (VIl). LRMS was by APCI+ and data quoted are for [M+H]⁺.

| Example | | Mass Spec. |
|---|---|---|
| Number | Structure | Data |
| 3 | 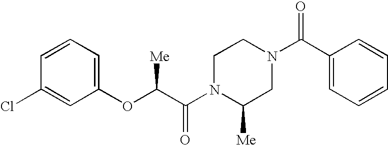 | 386 |
| 4 | 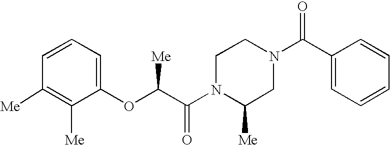 | 380 |
| 5 | 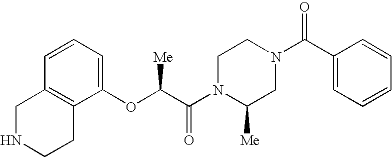 | 408 |
| 6 | 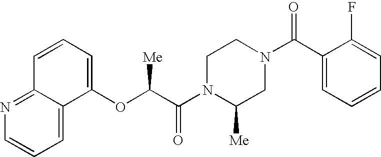 | 422 |
| 7 | 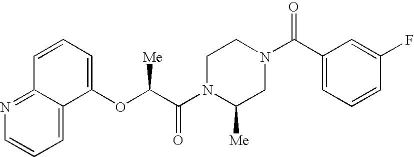 | 422 |
| 8 | 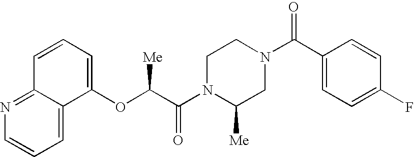 | 422 |
| 9 | 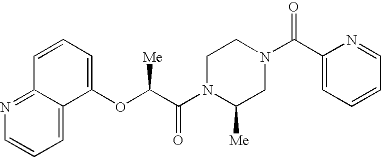 | 405 |
| 10 | 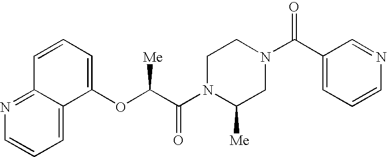 | 405 |
| 11 | 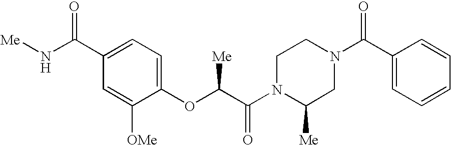 | 440 |
| 12 | 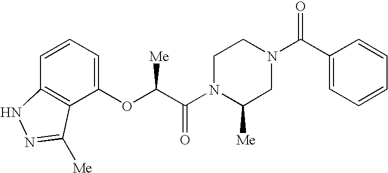 | 407 |

Examples 13-45 were prepared by the method described above for Example 2, using the corresponding compound of formula (II) and R⁵OH. LRMS was by APCI+ and data quoted are for [M+H]⁺, unless otherwise stated.

| Example | | Mass Spec. |
|---|---|---|
| Number | Structure | Data |
| 13 | 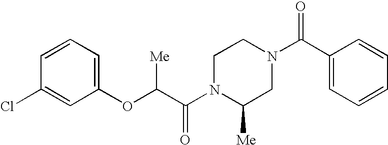 | 386 |
| 14 | 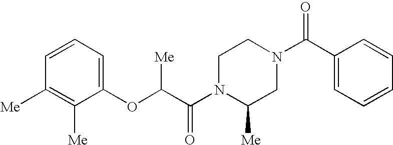 | 380 |
| 15 | 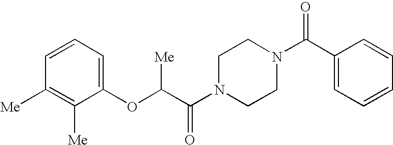 | 366 |
| 16 | 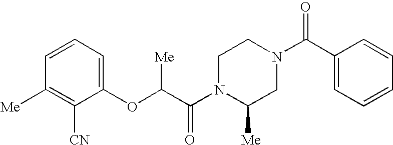 | 391 |
| 17 | 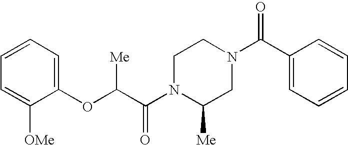 | 383 |
| 18 | 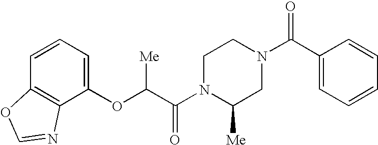 | 393 |
| 19 | 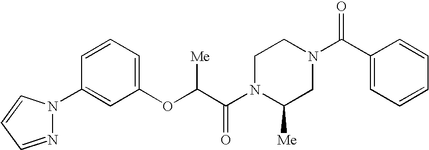 | 419 |
| 20 | 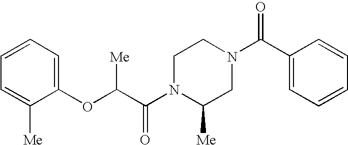 | 367 |
| 21 | 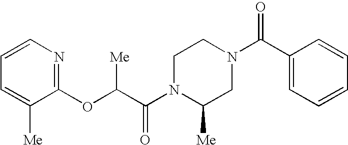 | 368 |
| 22 | 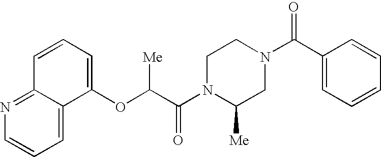 | 403 (ESI+, M⁺) |
| 23 | 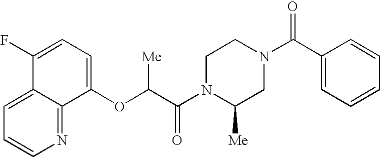 | 421 |
| 24 | 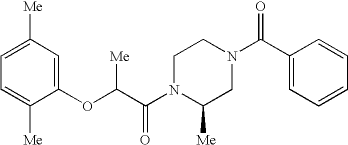 | 380 (ESI+, M⁺) |
| 25 | 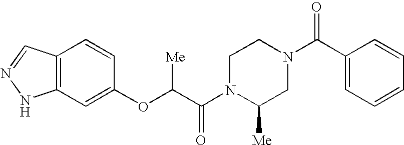 | 393 |
| 26 | 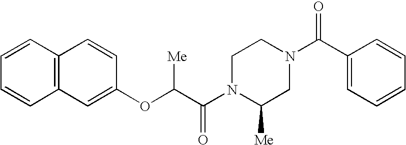 | 402 |
| 27 | 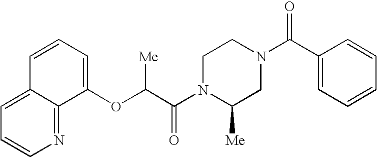 | 403 |
| 28 | 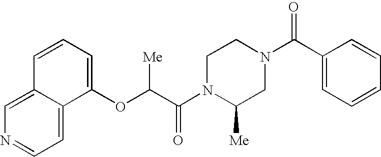 | 403 |
| 29 | 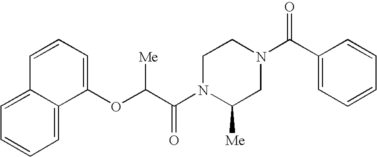 | 402 |
| 30 | 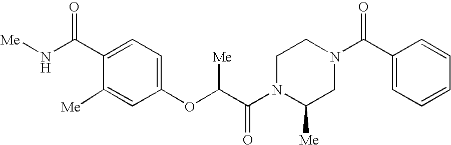 | 424 |
| 31 | 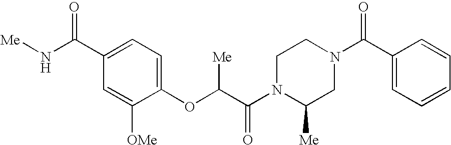 | 440 |
| 32 | 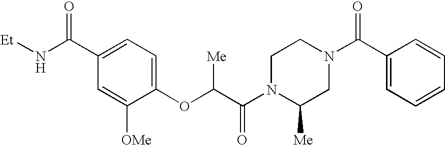 | 454 |
| 33 | 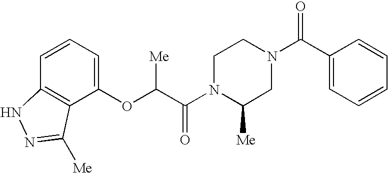 | 407 |
| 34 | 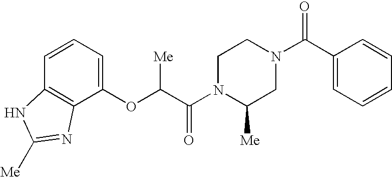 | 407 |
| 35 | 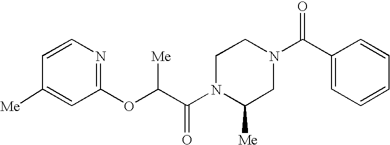 | 368 |
| 36 | 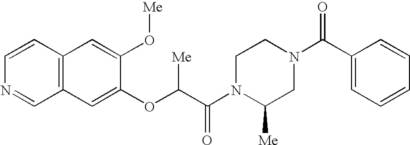 | 434 |
| 37 | 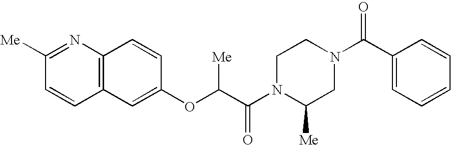 | 417 |
| 38 | 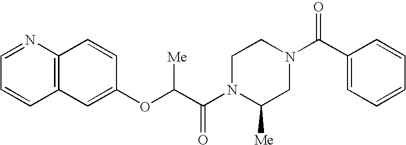 | 403 |
| 39 | 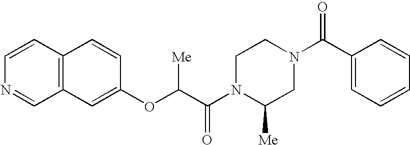 | 403 |
| 40 | 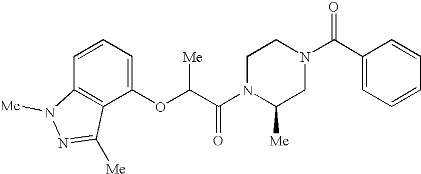 | 421 |
| 41 | 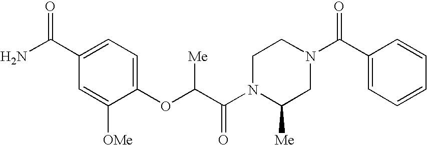 | 426 |
| 42 | 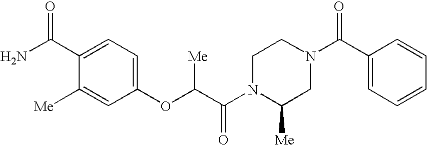 | 410 |
| 43 | 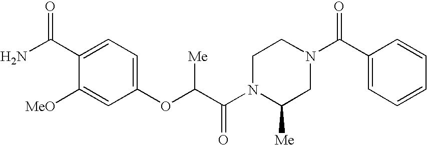 | 426 |
| 44 | 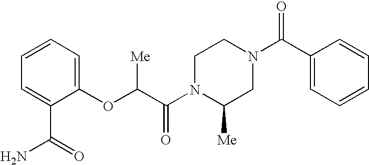 | 396 |
| 45 | 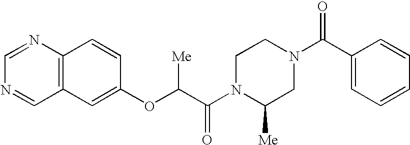 | 405 |

### EXAMPLE 46

### (2S)-1-[(2R)-4-Benzoyl-2-methyl-piperazin-1-yl]-2-[2-methoxy-4-(2H-[1,2,4]triazol-3-yl)-phenoxy]-propan-1-one

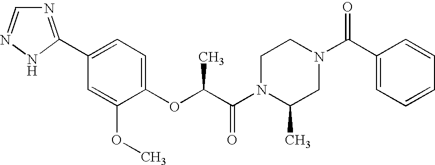

The compound of Preparation 10 (125 mg, 0.26 mmol) was added portionwise to a stirred solution of hydrazine monohydrate (25 μL, 0.52 mmol) in glacial acetic acid (4 mL). The resulting solution was heated at 90° C. for 3 hours. The reaction mixture was then evaporated under reduced pressure and the residual colourless oil was partitioned between dichloromethane (50 mL) and saturated sodium hydrogen carbonate solution (8 mL). The organic phase was separated and the aqueous phase was re-extracted with dichloromethane. (30 mL). The combined organic phases were dried over magnesium sulfate and concentrated in vacuo to yield a colourless foam. The foam was dried under reduced pressure to afford the title compound in 94% yield, 110 mg.

LRMS (ES⁺): m/z [M+H]⁺ 450

### EXAMPLE 47

### 1-[(2R)-4-Benzoyl-2-methyl-piperazin-1-yl]-2-[2-methyl-3-(1-methyl-1H-pyrazol-3-yl)-phenoxy]-propan-1-one

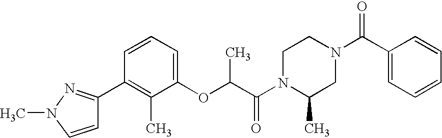

The compounds of Preparations 17 (100 mg, 0.53 mmol) and 25 (180 mg, 0.53 mmol) and cesium carbonate (172.7 mg, 0.53 mmol) in acetone (8 mL) were heated under reflux for 3.5 hours. The reaction mixture was then cooled and evaporated under reduced pressure. The residue was partitioned between dichloromethane (30 mL) and water (8 mL). The organic phase was separated and the aqueous phase was re-extracted with further dichloromethane (30 mL). The combined organic extracts were dried over magnesium sulfate and concentrated in vacuo to give a light brown oil. The oil was chromatographed using an Isolute® Flash silica column, eluting with dichloromethane:methanol, 99:1 to 97:3, to give a colourless oil. This oil was azeotroped with dichloromethane to afford the title compound as colourless foam in 89% yield, 210 mg.

LRMS (ES⁺): m/z [M+H]⁺ 447

### EXAMPLE 48

### 1-[(2R)-4-Benzoyl-2-methyl-piperazin-1-yl-2-(2-methyl-3-[1,2,4]oxadiazol-5-yl-phenoxy)-propan-1-one

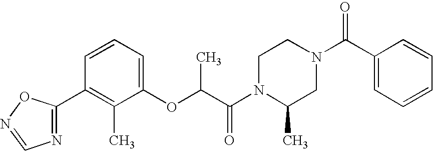

The compound of Preparation 29 (280 mg, 0.62 mmol) was heated in a mixture of glacial acetic acid (3 mL) and dioxane (3 mL) at 90° C., for 3.5 hours. The mixture was then allowed to cool and was evaporated under reduced pressure to give a yellow oil. The oil was dissolved in ethyl acetate (50 mL) and washed with 10% sodium carbonate solution (10 mL) and water (10 mL). The organic phase was dried over magnesium sulfate and concentrated in vacuo to give a colourless foam. The foam was purified on an Isolute® flash silica column, eluting with dichloromethane:methanol, 99:1 to 97:3, to give a colourless oil. The oil was azeotroped with dichloromethane to afford the title compound as a colourless foam in 44% yield, 118 mg.

LRMS (ES⁻): m/z [M−H]⁻ 433

### EXAMPLE 49

### (2S)-1-[(2R)-4-Benzoyl-2-methyl-piperazin-1-yl]-2-[3-(2H-pyrazol-3-yl)-phenoxy]-propan-1-one

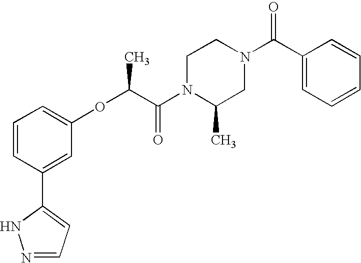

(3R)-(3-methyl-piperazin-1-yl)-phenyl-methanone [(114 mg, 0.56 mmol), J. Med. Chem. 43(23), 4499; 2000], 3-(diethoxyphosphoryloxy)-1,2,3-benzotriazin-4(3H)-one (167.5 mg, 0.56 mmol) and triethylamine (78 μL, 0.56 mmol) were added to a solution of the compound of Preparation 23 (130 mg, 0.56 mmol) in tetrahydrofuran (8 mL) and the resulting solution was stirred at room temperature for 48 hours. The mixture was then diluted with ethyl acetate (30 mL) and washed with water (10 mL), 10% citric acid (10 mL), water (10 mL) and sodium hydrogen carbonate solution. The organic phase was dried over magnesium sulfate and concentrated in vacuo. The residue was purified on an Isolute® flash silica column, eluting with dichloromethane:methanol, 98:2 to 96:4, to give a colourless foam. The foam was azeotroped with dichloromethane and dried under reduced pressure to afford the title compound in 85% yield, 198 mg.

LRMS (ES⁻): m/z [M−H]⁻ 417

### EXAMPLE 50

### (2S)-1-[(2R)-4-Benzoyl-2-methyl-piperazin-1-yl]-2-[2-methyl-3-(2H-pyrazol-3-yl)-phenoxyl-propan-1-one

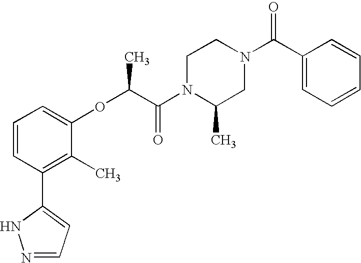

The title compound was prepared by the method described above for Example 49, using the compound of Preparation 24 and (3R)-(3-methyl-piperazin-1-yl)-phenyl-methanone (J. Med. Chem. 43(23), 4499; 2000).

LRMS (ES⁻): m/z [M−H]⁻ 431

### EXAMPLE 51

### 1-[(2R)-4-Benzoyl-2-methyl-piperazin-1-yl]-2-[4-(1H-imidazol-2-yl)-2-methoxy-phenoxy]-propan-1-one

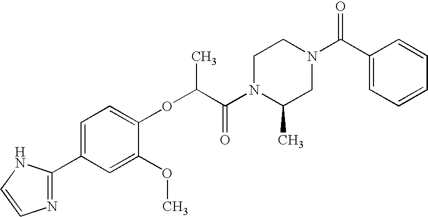

The compounds of Preparation 33 (30 mg, 0.16 mmol) and Preparation 25 (53.5 mg, 0.16 mmol) and cesium carbonate (51.3 mg, 0.16 mmol) in N,N-dimethylformamide (4 mL) were heated at 80° C. for 18 hours. The reaction mixture was allowed to cool and was then evaporated under reduced pressure and the residue was partitioned between dichloromethane (30 mL) and water (8 mL). The aqueous phase was separated and was re-extracted with dichloromethane (30 mL). The combined organic extracts were dried over magnesium sulfate and concentrated in vacuo. The residue was purified on an Isolute® flash silica column, eluting with, dichloromethane:methanol, 99:1 to 95:5, to afford the title compound as a beige foam in 74% yield, 52 mg.

LRMS (ES⁺): m/z [M+H]⁺ 449

### EXAMPLES 52 to 53

The following compounds, of the general formula shown below, were prepared by the method described above for Example 51, using the compound of Preparation 25 and the appropriate phenol.

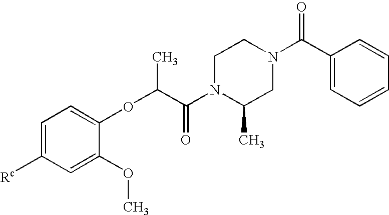

| No. | R^{c} | Data |
|---|---|---|
| 52 | 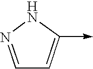 | LRMS (ES⁻): m/z [M − H]⁻ 447 |
| 53 | 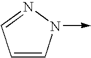 | LRMS (ES⁻): m/z [M + Na]⁺ 471 |

### EXAMPLES 54 to 57

The following compounds, of the general formula shown below, were prepared by the method described above for Example 51, using the compound of Preparation 25 and the appropriate phenol.

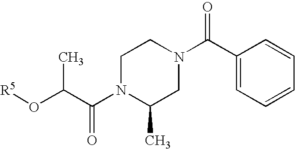

| | | LRMS (APCI⁺): m/z |
|---|---|---|
| No. | R⁵ | [M + H]⁺ |
| 54 | 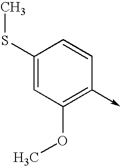 | 429 |
| 55 | 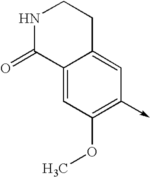 | 452 |
| 56 | 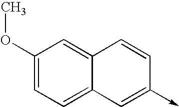 | 433 |
| 57 | 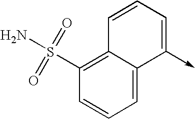 | 482 |

Example 54: phenol prepared as described in Preparation 67

Example 55: phenol prepared as described in Preparation 68

Example 56: 6-methoxy-2-naphthol is commercially available

Example 57: 5-hydroxy-1-naphthalene sulfonamide is commercially available

### EXAMPLE 58

### 1-[(2R)-4-Benzoyl-2-methyl-piperazin-1-yl]-2-(4-methanesulfonyl-2-methoxy-phenoxy)-propan-1-one

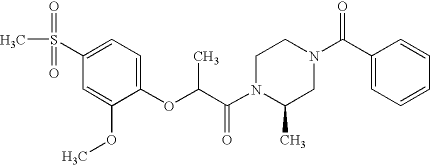

A solution of oxone (215 mg, 0.35 mmol) in water (2 mL) was added to a solution of the compound of Example 54 (100 mg, 0.23 mmol) in methanol (4 mL) and the mixture was stirred for 18 hours at room temperature. The solvent was then evaporated under reduced pressure and the residue was partitioned between water (20 mL) and dichloromethane (20 mL). The aqueous layer was separated and re-extracted with dichloromethane (30 mL). The combined organic extracts were dried over magnesium sulfate and concentrated in vacuo. The residue was purified on an Isolute® flash silica column, eluting with dichloromethane:methanol, 95:5 to afford the title compound as a white solid in 76% yield, 80 mg.

LRMS (APCI⁺): m/z [M+H]⁺ 461

### EXAMPLE 59

### N-{4-[2-(2R)-(4-Benzoyl-2-methyl-piperazin-1-yl)-1-methyl-2-oxo-ethoxy]-3-methoxy-phenyl}-methanesulfonamide

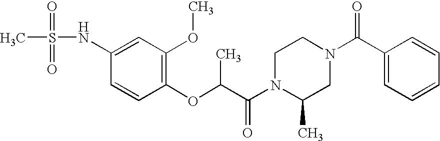

Triethylamine (0.08 mL, 0.57 mmol) was added to a solution of the compound of Preparation 30 (150 mg, 0.38 mmol) in dichloromethane (3 mL) and the mixture was cooled in an ice bath. Methanesufonyl chloride (0.03 mL, 0.42 mmol) was added dropwise and the reaction mixture was allowed to stir at room temperature for 18 hours. The mixture was then diluted with further dichloromethane (20 mL) and washed with water. The organic phase was dried over magnesium sulfate and concentrated in vacuo. The residue was then purified by column chromatography on silica gel, eluting with ethyl acetate:methanol, 98:2, to afford the title compound as a white solid in 49% yield, 88 mg.

LRMS (APCI⁺): m/z [M+H]⁺ 476

### EXAMPLE 60

### (2S)-1-[(2R)-4-Benzoyl-2-methyl-piperazin-1-yl]-2-(2-chloro-quinolin-5-yloxy)-propan-1-one

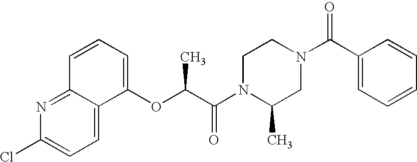

A solution of the compound of Preparation 46 (1.8 g, 4.3 mmol) in phosphorous oxychloride (5 mL) was heated at 100° C. for two hours. The reaction mixture was then cooled to room temperature and evaporated under reduced pressure. The residue was dissolved in dichloromethane (20 mL) and washed with saturated aqueous sodium hydrogen carbonate solution (10 mL) and water (2×10 mL). The aqueous phase was basified with 1M sodium hydroxide solution and was re-extracted with ethyl acetate. The combined organic extracts were then dried over magnesium sulfate and concentrated in vacuo. The residue was purified by column chromatography on silica gel, eluting with pentane:ethyl acetate 20:80, 0:100 to afford the title compound as a pale yellow solid in 31% yield, 580 mg.

¹H NMR (400 MHz, CDCl₃) δ: 1.00-1.40(m, 3H), 1.70-1.80(m, 3H), 2.80-4.80 (m, 7H), 5.20(m, 1H), 6.90(m, 1H), 7.20-7.45(m, 6H), 7.55-7.70 (m, 2H), 8.55(d, 1H)

LRMS (APCI₊): m/z [M+H]⁺ 438

### EXAMPLES 61 to 62

A mixture of the compound of Example 60 (30 mg, 0.069 mmol), tetrabutyl ammonium fluoride (18 mg, 0.14 mmol), triethylamine (96 μL, 0.69 mmol) and the appropriate amine (0.69 mmol) in dimethylsulfoxide (1 mL) was heated at 100° C. for 24 hours. The reaction mixtures were then purified directly by HPLC using a Phenomenex Luna C18 system, eluting with water/lacetonitrile/trifluoroacetic acid (5:95:0.1):acetonitrile, 95:5 to 5:95, to afford the desired compounds of general formula shown below:

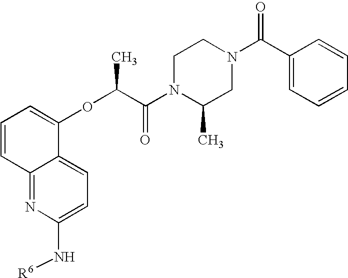

| No. | R⁶ | LRMS (APCI⁺): m/z [M − H]⁺ |
|---|---|---|
| 61 | 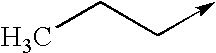 | 461 |
| 62 | 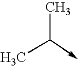 | 461 |

### EXAMPLE 63

### 1-[(2R)-4-Benzoyl-2-methyl-piperazin-1-yl)-2-(1-chloro-isoquinolin-5-yloxy)-propan-1-one

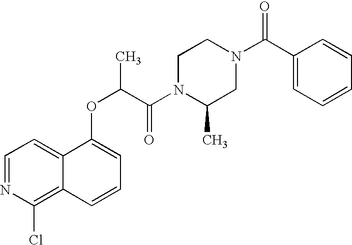

The title compound was prepared by the method described above for Example 60, using the compound of Preparation 63 and phosphorus oxychloride.

LRMS (APCI+): m/z [M+H]⁺ 438

### EXAMPLES 64 to 65

A mixture of the compound of Example 63 (45 mg, 0.1 mmol), tetrabutyl ammonium fluoride (27 mg, 0.2 mmol), triethylamine (140 82 L, 1 mmol) and the appropriate amine (1 mmol) in dimethylsulfoxide (1 mL) was heated at 130° C. for 24 hours. The reaction mixtures were then purified directly by HPLC using a Phenomenex Luna C18 system, eluting with water/acetonitrile/trifluoroacetic acid (5:95:0.1):acetonitrile, 95:5 to 5:95 to afford the desired compounds of general formula shown below:

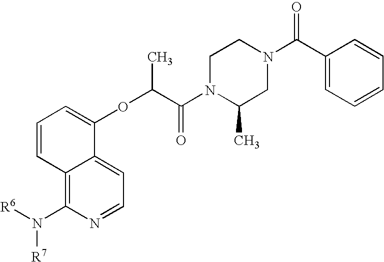

| No. | R⁶ | R⁷ | LRMS (APCI⁺): m/z [M − H]⁺ |
|---|---|---|---|
| 64 | 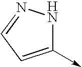 | H | 485 |
| 65 | CH₃ | CH₃ | 447 |

### EXAMPLE 66

### (2S)-2-(2-Amino-quinolin-5-yloxy)-1-[(2R-4-benzoyl-2-methyl-piperazin-1-yl]-propan-1-one

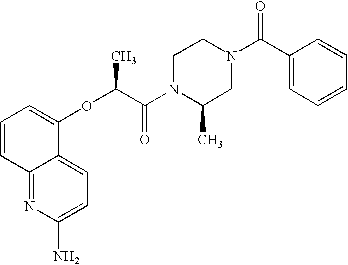

10% Pd/C (cat) and ammonium formate (10 mg, 0.15 mmol) were added to a solution of the compound of Preparation 49 (15 mg, 0.03 mmol) in ethanol (1 mL) and the mixture was heated at 70° C. for 2 hours. Additional ammonium formate (10 mg, 0.15 mmol) and Pd(OH)₂ (cat) were added and the reaction mixture was heated under reflux for a further 18 hours. The mixture was then cooled and filtered through Arbocel®, washing through with ethanol (10 mL). The filtrate was concentrated in vacuo and the residue was purified by HPLC using a Phenomenex Luna C18 system, eluting with water/acetonitrile/trifluoroacetic acid (5:95:0.1):acetonitrile, 95:5 to 5:95, to afford the title compound in 77% yield, 9.7 mg.

LRMS (ES⁺): m/z [M+H]⁺ 419

### EXAMPLE 67

### (2S)-1-[(2R)-4-Benzoyl-2-methyl-piperazin-1-yl]-2-(8-chloro-quinolin-5-yloxy)-propan-1-one

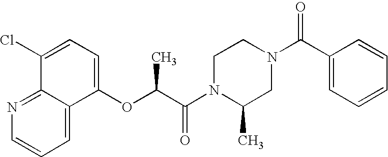

A mixture of the compound of Preparation 42 (30 mg, 0.07 mmol) and N-chlorosuccinimide (11 mg, 0.08 mmol) in acetonitrile (3 mL) was heated at 40° C. for 48 hours. The solvent was then evaporated under reduced pressure and the residue was dissolved in ethyl acetate (5 mL) and washed with water (2×5 mL). The organic phase was dried over magnesium sulfate and concentrated in vacuo to give a yellow residue. The residue was purified by HPLC using a Phenomenex Luna C18 system, eluting with water/acetonitrile/trifluoroacetic acid (5:95:0.1):acetonitrile, 95:5 to 5:95, to afford the title compound as a solid in 32% yield, 9.7 mg.

LRMS (APCI+): m/z [M+H]⁺ 438

### EXAMPLES 68 to 69

The following compounds, of the general formula shown below, were prepared by the method described above for Example 67, using the appropriate starting material and N-chlorosuccinimide.

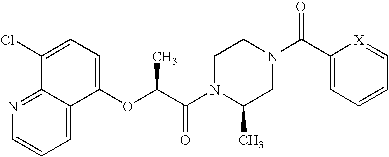

| No. | X | LRMS (APCI⁺): m/z [M − H]⁺ |
|---|---|---|
| 68 | N | 439 |
| 69 | C—F | 456 |

Examples 68 and 69 were purified by column chromatography on silica gel, eluting with dichloromethane:methanol, 99:1 to 96:4

### EXAMPLES 70 and 71

The compounds were prepared from the compound of Preparation 60 and 1-chloromethyl-4-fluoro-1,4-diazoniabicyclo[2.2.2]octane bis(tetrafluoroborate), using a similar preparation to Example 67. The compounds were purified by HPLC using a Phenomenex Luna C18(2) column 150×15 mm (10 micron particle size, 100 521 porosity), using a 2 solvent eluent of acetonitrile:water:trifluoroacetic acid (5:95:0.1) [solvent A] and acetonitile [solvent B]. A solvent gradient is run at a flow-rate of 20 ml/min as in the table below.

| Time (min) | % B |
|---|---|
| 0 | 5 |
| 0.6 | 5 |
| 9.5 | 95 |
| 10.5 | 95 |
| 10.6 | 5 |
| 12 | 5 |

| | | HPLC retention | LRMS (APCI⁺): |
|---|---|---|---|
| No | Structure | time (min.) | m/z [M + H]⁺ |
| 70 | 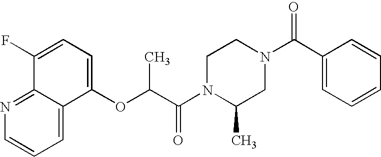 | 4.99 | 422 |
| 71 | 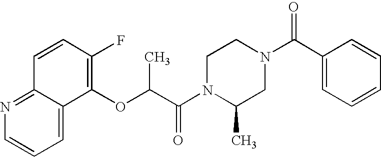 | 5.77 | 422 |

### EXAMPLE 72

### (2S)-1[(2R)-4-Benzoyl-2-methyl-piperazin-1-yl]-2-(8-methyl-quinolin-5-yloxy)-propan-1-one

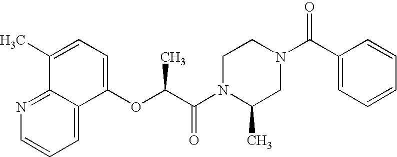

A mixture of the compound of Preparation 44 (200 mg, 0.4 mmol), methyl boronic acid (74 mg, 1.2 mmol), bis(triphenylphosphine)palladium(II) chloride (29 mg, 0.04 mmol) and potassium carbonate (229 mg, 1.6 mmol) in N,N-dimethylformamide (4 mL) was heated at 100° C. for 18 hours. TIc analysis showed that the reaction was incomplete and so additional amounts of methyl boronic acid (74 mg) and bis(triphenylphosphine)palladium(II) chloride (29 mg) were added, and the mixture was allowed to stir for a further 24 hours at 100° C. The reaction mixture was then filtered through Arbocel®, washing through with ethyl acetate (25 mL) and water (3×20 mL). The layers were separated and the aqueous layer was re-extracted with ethyl acetate. The combined organic solutions were dried over magnesium sulfate and concentrated in vacuo to afford a yellow residue. The residue was purified by HPLC using a Phenomenex Luna C18 system, eluting with water/acetonitrile/trifluoroacetic acid (5:95:0.1):acetonitrile, 95:5 to 5:95. The appropriate fraction was then azeotroped with diethyl ether to afford the title compound as a yellow solid in 19% yield, 33 mg.

LRMS (APCI⁺): m/z [M+H]⁺ 418

### EXAMPLE 73

### (2S)-1-[(2R)-4-Benzoyl-2-methyl-piperazin-1-yl]-2-[8-(2H-pyrazol-3-yl)-quinolin-5-yloxy]-propan-1-one

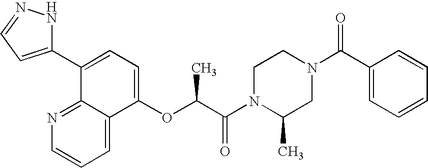

4M Hydrochloric acid (2 mL) was added to a suspension of the compound of Preparation 45 (43 mg, 0.07 mmol) in methanol (2 mL), and the resulting solution was stirred at room temperature for 18 hours. The reaction mixture was then evaporated under reduced pressure and the residue was dissolved in water and washed twice with dichloromethane. The combined organic layers were dried over magnesium sulfate and concentrated in vacuo to give a yellow residue. The residue was purified by column chromatography on silica gel, eluting with dichloromethane:methanol, 98:2, to afford the title compound as a yellow solid in 68% yield, 23 mg

LRMS (APCI⁺): m/z [M+H]⁺ 470

### EXAMPLE 74

### (2S)-1-[(2R)-4-Benzoyl-2-methyl-piperazin-1-yl]-2-[2-(2H-pyrazol-3-ylamino)-quinolin-5-yloxy]-propan-1-one

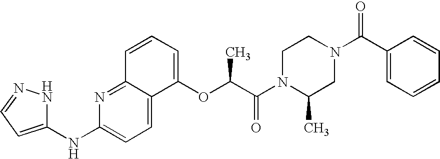

A mixture of the compound of Example 60 (50 mg, 0.11 mmol), 3-aminopyrazole (29 mg, 0.33 mmol) and triethylamine (48 μL, 0.33 mmol) in 2-propanol (0.5 mL) was heated at 90° C. in a Reactivial™. After 18 hours, cesium fluoride (33 mg, 0.22 mmol) was added and the mixture was heated at 100° C. for a further 48 hours. The reaction mixture was then filtered and concentrated in vacuo to afford an oily residue. The residue was purified by HPLC using a Phenomenex Luna C18 system, eluting with water/acetonitrile/trifluoroacetic acid (5:95:0.1):acetonitrile, 95:5 to 5:95, to afford the title compound as a yellow solid 3 mg.

LRMS (APCI⁺): m/z [M+H]⁺ 485

### EXAMPLE 75

### 5-{(1S)-2-[(2R)-4-Benzoyl-2-methyl-piperazin-1-yl]-1-methyl-2-oxo-ethoxy}-1H-quinolin-2-one

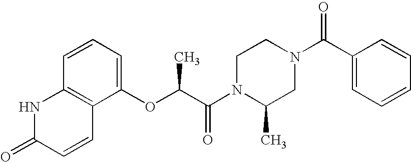

The compound of Example 60 (200 mg, 0.46 mmol) and acetic acid (3 mL) were dissolved in water and the solution was heated under reflux for 24 hours. The reaction mixture was then diluted with water and washed with dichloromethane (3×10 mL). The combined organic extracts were dried over magnesium sulfate and concentrated in vacuo to give a yellow oil. The oil was purified by column chromatography on silica gel, eluting with dichloromethane:methanol, 95:5 to afford the title compound as pale yellow solid in 40% yield, 76 mg.

LRMS (APCI⁺): m/z [M+H]⁺ 418

### EXAMPLE 76

### 5-{(1S)-2-[(2R)-4-Benzoyl-2-methyl-piperazin-1-yl]-1-methyl-2-oxo-ethoxy}-8-chloro-1H-quinolin-2-one

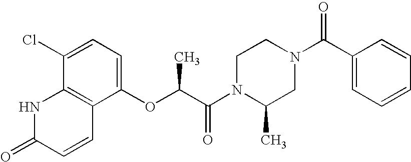

The title compound was prepared by the method described above for Example 67, using the compound of Example 75 and N-chlorosuccinimide.

LRMS (APCI⁺): m/z [M+H]⁺ 454

### EXAMPLE 77

### (2S)-1-[(2R)-4-Benzoyl-2-methyl-piperazin-1-yl]-2-(2-methoxy-quinolin-5-yloxy)-propan-1-one

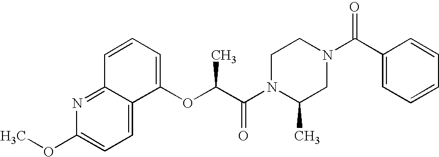

A mixture of the compound of Example 60 (20 mg, 0.04 mmol) and sodium methoxide (0.5M in methanol, 0.7 mL) in N,N-dimethylformamide (0.5 mL) was heated in a Reactivial™ at 70° C. for 6 hours. The reaction mixture was cooled, and evaporated under reduced pressure. The residue was dissolved in dichloromethane (5 mL) and washed with brine (5 mL) and water (2×5 mL). The organic phase was dried over magnesium sulfate and concentrated in vacuo. The residue was dissolved in dimethylsulfoxide (0.5 mL) and was purified by HPLC using a Phenomenex Luna C18 system, eluting with water/acetonitrile/trifluoroacetic acid (5:95:0.1):acetonitrile, 95:5 to 5:95. The appropriate fraction was then azeotroped with diethyl ether to afford the title compound as a yellow solid in 40% yield, 7 mg.

LRMS (APCI⁺): m/z [M+H]⁺ 434

### EXAMPLE 78

### 5-{(1S)-2-[(2R)-4-Benzoyl-2-methyl-piperazin-1-yl]-1-methyl-2-oxo-ethoxy]-isoquinoline-1-carboxylic acid methyl ester

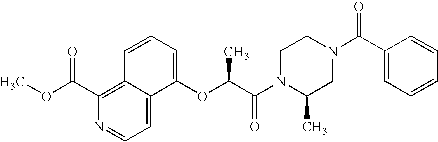

The compound of Preparation 65 (80 mg, 0.18 mmol), dichloro-bis(triphenylphosphine)palladium (15 mg, 0.018 mmol) and triethylamine (51 μL, 0.37 mmol) were dissolved in methanol (5 mL) and transferred to a sealed vessel. The vessel was heated to 100° C. and the mixture was stirred under 100 psi of carbon monoxide gas for 42 hours. The reaction mixture was then filtered through Arbocel®, washing through with methanol and the filtrate was concentrated in vacuo. The residue was purified on an Isolute® flash silica column, eluting with dichloromethane:methanol, 99:1 to afford the title compound as a white foam in 43% yield, 36 mg.

LRMS (APCI⁺): m/z [M+H]⁺ 462

### EXAMPLE 79

### 5-{2-[(2R)-4-Benzoyl-2-methyl-piperazin-1-yl]-1-methyl-2-oxo-ethox}-quinoline-2-carboxylic acid methylamide

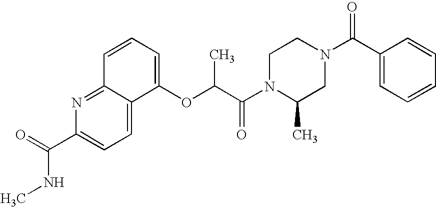

A mixture of the compound of Preparation 48 (20 mg, 0.05 mmol), methylamine (33% in ethanol, 30 mg, 0.5 mmol), O-(1H-benzotriazol-1-yl)-N,N,N′,N′-tetramethyluronium hexafluorophosphate (2 mg, 0.08 mmol) and triethylamine (62 μL, 0.5 mmol) in dichloromethane (1 mL) was stirred at room temperature for 18 hours. Additional methylamine (30 mg, 0.5 mmol) and triethylamine (62 μL, 0.5 mmol) were added to the reaction mixture and stirring continued for a further 18 hours. The reaction mixture was then diluted with dichloromethane and washed with water (2×5 mL) and brine (5 mL). The organic phase was dried over magnesium sulfate and concentrated in vacuo to give a yellow residue. The residue was purified by column chromatography on silica gel, eluting with dichloromethane:methanol, 96:4, to afford the title compound as a white solid in 13% yield, 3 mg.

LRMS (APCI⁺): m/z [M+H]⁺ 461

### EXAMPLE 80

### 5-{(1S)-2-[(2R)-4-Benzoyl-2-methyl-piperazin-1-yl]-1-methyl-2-oxo-ethoxy}isoquinoline-1-carboxylic acid methylamide

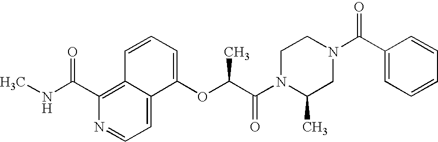

The title compound was prepared by the method described above for Example 79, using the compound of Example 78 and methylamine.

LRMS (APCI⁺): m/z [M+H]⁺ 461

### EXAMPLE 81

### 4-{(1S)-2-[(2R)-4-Benzoyl-2-methyl-piperazin-1-yl]-1-methyl-2-oxo-ethoxy}N-methyl-3-trifluoromethoxy-benzamide

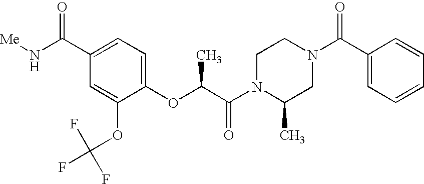

The compound of Preparation 59 (100 mg, 0.2 mmol) and methylamine (33% in ethanol, 4 mL) were heated at 90° C. in a Reactivial™ for 18 hours. The solvent was then evaporated under reduced pressure and the residue was purified by column chromatography on silica gel, eluting with dichloromethane:methanol, 99:1 to 97:3, to afford the title compound as a pale orange foam in quantitative yield, 100 mg.

LRMS (APCI⁺): m/z [M+H]⁺ 494

### EXAMPLE 82

### (2S)-1-[(2R)-4-Benzoyl-2-methyl-piperazin-1-yl]-2-(8-chloro-2-methylamino-quinolin-5-yloxy)-propan-1-one

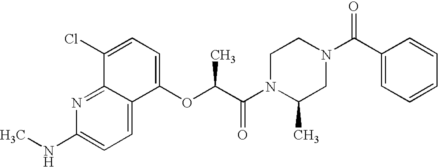

A mixture of the compound of Preparation 52 (60 mg, 0.12 mmol), methylamine hydrochloride (86 mg, 1.2 mmol), cesium fluoride (39 mg, 0.24 mmol) and triethylamine (0.18 mL, 1.2 mmol) in dimethylsulfoxide (1 mL) was heated in a microwave for 8 minutes at 170° C. The reaction, mixture was then filtered and the filtrate was purified by HPLC using a Phenomenex Luna C18 system, eluting with water/acetonitrile/trifluoroacetic acid (5:95:0.1):acetonitrile, 95:5 to 5:95. The appropriate fraction was then azeotroped with diethyl ether to afford the title compound as a brown solid in 19% yield, 10.8 mg.

LRMS (APCI⁺): m/z [M+H]⁺ 467

### EXAMPLE 83

### 1-[(2R)-4-Benzoyl-2-methyl-piperazin-1-yl]-2-(1-methylamino-isoquinolin-5-yloxy)-propan-1-one

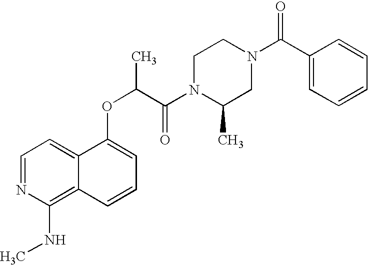

The title compound was prepared by the method described above for Example 82, using the compound of Example 63 and methylamine.

LRMS (APCI⁺): m/z [M+H]⁺ 433

### EXAMPLE 84

### (2S)-1-[(2R)-4-Benzoyl-2-methyl-piperazin-1-yl]-2-(8-chloro-2-dimethylamino-quinolin-5-yloxy)-propan-1-one

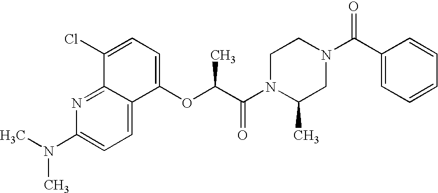

A mixture of the compound of Preparation 52 (50 mg, 0.1 mmol), dimethylamine (86 mg, 1 mmol), cesium fluoride (32 mg, 0.2 mmol) and triethylamine (0.15 mL, 1 mmol) in dimethylsulfoxide (4 mL) was heated at 170° C. in a Reactivial™ for 18 hours. The reaction mixture was then filtered and the filtrate was purified by HPLC using a Phenomenex Luna C₁₈ system, eluting with water/acetonitrile/trifluoroacetic acid (5:95:0.1):acetonitrile, 95:5 to 5:95, to afford the title compound as a white solid, 5 mg.

LRMS (APCI⁺): m/z [M+H]⁺ 481

### EXAMPLE 85

### (2S)-1-[(2R)-4-Benzoyl-2-methyl-piperazin-1-yl]-2-[1-(2H-pyrazol-3-ylamino)-isoquinolin-5-yloxy]-propan-1-one

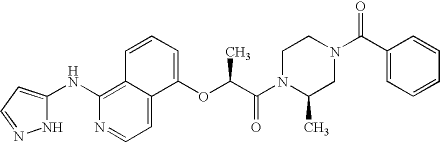

A mixture of the compound of Example 63 (45 mg, 0.1 mmol), tetrabutyl ammonium fluoride (27 mg, 0.2 mmol), triethylamine (0.14 mL, 1 mmol) and 3-aminopyrazole (83 mg, 1 mmol) in dimethylsulfoxide (1 mL) was heated at 100° C. for 24 hours. The reaction mixture was then purified directly by HPLC using a Phenomenex Luna C₁₈ system, eluting with water/acetonitrile/trifluoroacetic acid (5:95:0.1):acetonitrile, 95:5 to 5:95, to afford the title compound in 39% yield, 19 mg.

LRMS (APCI+): m/z [M+H]⁺ 485

### EXAMPLE 86

### (2S)-1-[(2R)-4-Benzoyl-2-methyl-piperazin-1-yl)]-2-(3.6-dimethyl-1H-indazol-4-yloxy)-propan-1-one

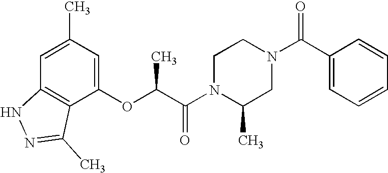

4M hydrochloric acid in dioxane (1 mL) was added to the compound of Preparation 77 (45 mg, 86 μmol) in methanol and the solution was stirred for 2 hours. The reaction mixture was evaporated under reduced pressure and the residue was dissolved in dichloromethane and basified with 1M sodium hydroxide solution to pH12. The mixture was then passed through a phase separation column and the filtrate was concentrated in vacuo. Purification by column chromatography on silica gel, eluting with dichloromethane:methanol:0.88 ammonia, 96:4:1, afforded the title compound as a pale pink foam in 59% yield, 20 mg.

LRMS (APCI⁺): m/z [M+H]⁺ 421

### EXAMPLE 87

### (2S)-1-[(2R)-2-Methyl-4-(pydridine-2-carbonyl)-piperazin-1-yl]-2-(quinolin-5-yloxy)-propan-1-one

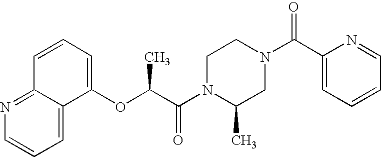

A mixture of the compound of Preparation 51 (150 mg, 0.5 mmol), picolinic acid (49 mg, 0.6 mmol) O-(1H-benzotriazol-1-yl)-N,N,N′,N′-tetramethyluronium hexafluorophosphate (190 mg, 0.75 mmol) and triethylamine (0.93 mL, 10 mmol) in dichloromethane (3 mL) was stirred for 18 hours at room temperature. The reaction mixture was then diluted with further dichloromethane and washed with sodium hydroxide (10 mL) and water (2×10 mL). The organic phase was dried over magnesium sulfate and concentrated in vacuo. Purification of the residue by column chromatography on silica gel, eluting with dichloromethane:methanol, 99:1 to 95:5, afforded the title compound as a white solid in 49% yield, 99 mg.

LRMS (APCI⁺): m/z [M+H]⁺ 405

### EXAMPLE 88

### (2S)-1-[4-(2-Fluoro-benzoyl)-(2R)-2-methyl-piperazin-1-yl]-2-(quinolin-5-yloxy)-propan-1-one

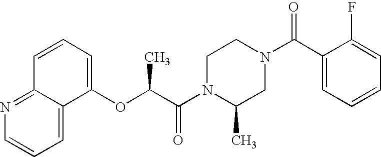

A mixture of the compound of Preparation 51 (150 mg, 0.5 mmol), 2-fluorobenzoyl chloride and triethylamine in dichloromethane (3 mL) was stirred at room temperature for 18 hours. The reaction mixture was then diluted with dichloromethane and washed with water (2×10 mL). The organic phase was dried over magnesium sulfate and concentrated in vacuo. Purification by column chromatography on silica gel, eluting with dichloromethane:methanol, 98:2, afforded the title compound as a white solid in 73% yield, 153 mg.

LRMS (APCI⁺): m/z [M+H]⁺ 422

### EXAMPLE 89

### 4-{(1S)-2-[(2R)-4-Benzoyl-2-methyl-piperazin-1-yl]-1-methyl-2-oxo-ethoxy}-3-methoxy-N-methyl-benzamide

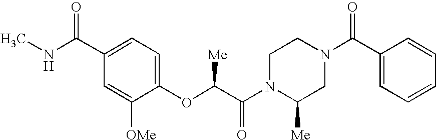

To a solution of (3R)-(3-methyl-piperazin-1-yl)-phenyl-methanone (J. Med. Chem. (2000), 43(23), 4499) (22 g, 0.11 mol) and (2S)-2-(2-methoxy-4-methylcarbamoyl-phenoxy)-propionic acid (25 g, 0.1 mol) (Preparation 5) in dry THF (250 ml) were added 1-N-hydroxybenzatriazole monohydrate (20 g, 0.13 mol), 1-ethyl-3-(3′dimethylaminopropyl)carbodiimide.HCl (17 g, 0.09 mol) and diisopropylethylamine (17 ml, 0.1 mol). After stirring for 1 hour a further portion of 1-ethyl-3-(3′dimethylaminopropyl)carbodiimide.HCl (17 g, 0.09 mol) was added followed by additional diisopropylethylamine (17 ml, 0.1 mol). After another hour a further portion of 1-ethyl-3-(3′dimethylaminopropyl)carbodiimide.HCl (17 g, 0.09 mol) was added followed by another portion of diisopropylethylamine (17 ml, 0.1 mol). The mixture was then stirred for a further 16 hours. After this time LCMS analysis indicated that all of the propionic acid had been consumed. The reaction mixture was evaporated to give a viscous oil which was treated with water (200 ml) followed by the slow addition of concentrated aqueous hydrochloric acid until the mixture was pH 3. The resulting off-white precipitate was filtered off and washed with water (3×100 ml). The solid was twice re-crystallised from ethanol:water (1:1 by volume, 120 ml) to afford the title compound as a white solid (32 g).

¹H NMR (400 MHz, CDCl₃): δ: 7.50-7.30 (6H, m), 7.27 (1H, m), 6.9 (1H, m) 6.08 (1H, br s), 5.1 (11H, br m), 4.90-2.80 (7H, m), 3.90 (3H, br s), 3.00 (3H, br s), 2.40 (3H, s), 1.70 (3H, br d), 1.40-0.9 (3H, m) ppm. LRMS (ESI+): m/z [M+H]⁺ 440 [α]_{D} −26.9° (1 mg/ml in MeOH, 25° C., wavelength 589 nM)

### EXAMPLE 90

### 5-{(1S)-2-[(2R)-4-Benzoyl-2-methyl-piperazin-1-yl]-1-methyl-2-oxo-ethoxy}-4-methoxy-pyridine-2-carboxylic acid methyl ester

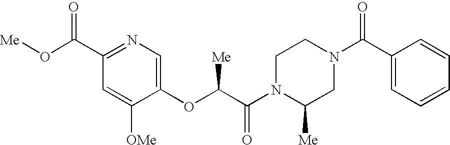

Di-tert-butyl azodicarboxylate (0.14 g, 0.6 mmol) and polymer supported triphenylphosphine (0.25 g, 0.75 mmol) were added to a stirred solution of (2R)-1-[(2R)-4-Benzoyl-2-methyl-piperazin-1-yl)-2-hydroxy-propan-1-one (Preparation 76) (0.083 g, 0.3 mmol) and 5-hydroxy-4-methoxy-pyridine-2-carboxylic acid methyl ester (0.083 g, 0.45 mmol) (Tetrahedron Letters, 38, 1297 (1997)) in dichloromethane (3 ml) at 0° C. After one hour the reaction mixture was allowed to warm to room temperature and additional di-tert-butyl azodicarboxylate (0.14 g, 0.6 mmol) and polymer supported triphenylphosphine (0.25 g, 0.75 mmol) were added. After 18 hours at room temperature the reaction mixture was filtered through Arbocel®, washing through with dichloromethane (10 mL). The filtrate was washed with saturated aqueous sodium hydrogencarbonate solution (20 ml), then the organic phase separated, dried (MgSO₄) and evaporated under reduced pressure. Purification of the residue by column chromatography on silica gel, eluting with ethyl acetate:pentane, 80:20, then ethyl acetate and finally ethyl acetate:methanol, 95:5, to afford the title compound as a white solid, 0.1 g.

LRMS (APCI⁺): m/z [M+H]⁺ 442

### EXAMPLE 91

### 5-{(1S)-2-[(2R)-4-Benzoyl-2-methyl-piperazin-1-yl]-1-methyl-2-oxo-ethoxy}-4-methoxy-pyridine-2-carboxylic acid methylamide

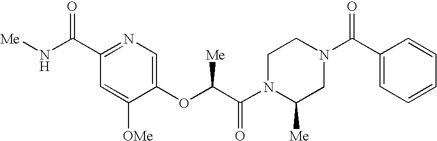

A solution of 5-{(1S)-2-[(2R)-4-benzoyl-2-methyl-piperazin-1-yl]-1-methyl-2-oxo-ethoxy}-4-methoxy-pyridine-2-carboxylic acid methyl ester (Example 90) (0.2 g, 0.45 mmol) in a 33% w/w solution of methylamine in ethanol (3 ml) was heated in a Reacti-vial™ at 50° C. for 18 hours. After cooling to room temperature solvent was evaporated under reduced pressure. The residue was purified by column chromatography on silica gel, eluting with dichloromethane:methanol:NH₃, 98:2:0.5 to 95:5:0.5, to afford the title compound as a white solid, 0.18 g.

¹H NMR (400 MHz, CD₃OD): δ: 8.05 (1H, S), 7.75 (1H, S), 7.50-7.40 (5H, m), 5.40 (1H, m), 4.60-3.00 (8H, m), 3.95 (3H, br s), 2.90 (3H, s), 1.60-1.00 (6H, m) ppm. Found C, 61.65; H, 6.47; N, 12.41. C₂₃H₂₈N₄O₅. 0.1 mol CH₂Cl₂ requires C, 61.80; H, 6.33; N, 12.48%. LRMS (APCI⁺): m/z [M+H]⁺ 441 [α]_{D} −15.5° (1 mg/ml in MeOH, 25° C., wavelength 589 nM)

### EXAMPLE 92

### 5-{(1S)-2-[(2R)-4-Benzoyl-2-methyl-piperazin-1-yl]-1-methyl-2-oxo-ethoxy}-4-methoxy-pyridine-2-carboxylic acid amide

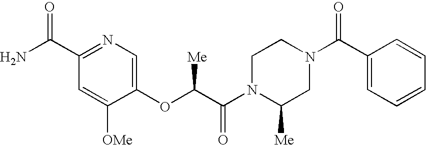

A solution of 5-{(1S)-2-[(2R)-4-benzoyl-2-methyl-piperazin-1-yl]-1-methyl-2-oxo-ethoxy}-4-methoxy-pyridine-2-carboxylic acid methyl ester (Example 90) (0.2 g, 0.45 mmol) in concentrated aqueous ammonia solution (2 ml) and methanol (1 ml) was heated in a Reacti-vial™ at 50° C. for 18 hours. After cooling to room temperature solvent was evaporated under reduced pressure. The residue was purified by column chromatography on silica gel, eluting with dichloromethane:methanol:NH₃, 98:2:0.5 to 95:5:0.5, to afford the title compound as a white solid, 0.16 g.

¹H NMR (400 MHz, CD₃OD): δ: 8.05 (1H, s), 7.75 (1H, s), 7.50-7.40 (5H, m), 5.40 (1H, m), 4.60-3.00 (9H, m), 3.95 (3H, br s), 1.60-1.00 (6H, m) ppm. Found C, 59.67; H, 6.11; N, 12.49. C₂₂H₂₆N₄O₅. 0.25 mol CH₂Cl₂ requires C, 59.78; H, 5.97; N, 12.54%.

LRMS (APCI⁺): m/z [M+H]⁺ 427

### EXAMPLE 93

### 5-{(1S)-2-[(2R)-4-Benzoyl-2-methyl-piperazin-1-yl]-1-methyl-2-oxo-ethoxy}-4-methoxy-pyridine-2-carboxylic acid ethylamide

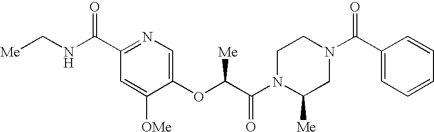

A solution of 5-{(1S)-2-[(2R)-4-benzoyl-2-methyl-piperazin-1-yl]-1-methyl-2-oxo-ethoxy}-4-ethoxy-pyridine-2-carboxylic acid methyl ester (Example 90) (0.15 g, 0.34 mmol) in ethylamine (1 ml) was heated in a Reacti-vial™ at 50° C. for 18 hours. After cooling to room temperature solvent was evaporated under reduced pressure. The residue was purified by column chromatography on silica gel, eluting with dichloromethane:methanol 96:4, to afford the title compound as a white solid, 0.14 g.

¹H NMR (400 MHz, CD₃OD): δ: 8.05 (1H, s), 7.80 (1H, s), 7.50-7.40 (5H, m), 5.40 (1H, m) 4.60-3.00 (8H, m), 4.00 (3H, br s), 3.45 (2H, q), 1.60-1.00 (6H, m), 1.20 (3H, t) ppm. Found C, 60.89; H, 6.72; N, 11.93. C₂₄H₃₀N₄O₅. 0.25 mol CH₂Cl₂ requires C, 61.22; H, 6.46; N, 11.78%.

LRMS (APCI⁺): m/z [M+H]⁺ 455

### EXAMPLE 94

### 5-{(1S)-2-[(2R)-4-Benzoyl-2-methyl-piperazin-1-yl]-1-methyl-2-oxo-ethoxy}-4-methoxy-pyridine-2-carboxylic acid cyclopropylamide

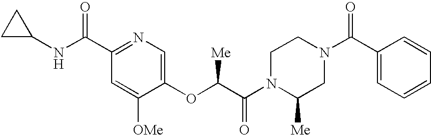

A solution of 5-{(1S)-2-[(2R)-4-benzoyl-2-methyl-piperazin-1-yl]-1-methyl-2-oxo-ethoxy}-4-methoxy-pyridine-2-carboxylic acid methyl ester (Example 90) (0.12 g, 0.27 mmol) in cyclopropylamine (1 ml) was heated in a Reacti-vial™ at 50° C. for 18 hours. After cooling to room temperature solvent was evaporated under reduced pressure. The residue was purified by column chromatography on silica gel, eluting with dichloromethane:methanol 95:5, to afford the title compound as a white solid, 0.09 g.

¹H NMR (400 MHz, CD₃OD): δ: 8.05 (1H, s), 7.70 (1H, s), 7.50-7.40 (5H, m), 5.40 (1H, m), 4.60-3.00 (8H, m), 3.95 (3H, br s), 2.85 (1H, m), 1.60-1.00 (6H, m), 0.8 (2H, m), 0.6 (2H, m) ppm. Found C, 63.02; H, 6.63; N, 11.83. C₂₅H₃₀N₄O₅. 0.13 mol CH₂Cl₂ requires C, 63.20; H, 6.39; N, 11.73%.

LRMS (APCI⁺): m/z [M+H]⁺ 467

### Preparation 1

### (2S)-2-(Quinolin-5-yloxy)-propionic acid methyl ester

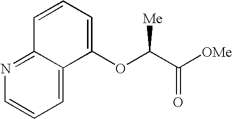

Triphenylphosphine (2.17 g, 8.27 mmol) was added to a stirred suspension of di-isopropyl azodicarboxylate (1.47 ml, 7.56 mmol) in THF (25 ml) at −5° C. 5-Hydroxyquinoline (1.0 g, 6.90 mmol) and methyl (R)-lactate (0.66 ml, 6.90 mmol) were then added and the solution allowed to stir at room temperature for 14 hours. The reaction mixture was diluted with ethyl acetate (50 ml), washed with water (30 ml), saturated potassium carbonate solution (30 ml) and brine (30 ml). The solution was dried (MgSO₄) and solvent evaporated under reduced pressure. The crude product was purified by column chromatography on silica gel eluting with a gradient system of pentane:ethyl acetate (4:1) changing to pentane:ethyl acetate (3:2). The title compound was obtained as a white solid (1.30 g).

LRMS (APCI+): m/z [M+H]⁺ 232.

### Preparation 2

### (2S)-2-(Quinolin-5-yloxy)-propionic acid sodium salt

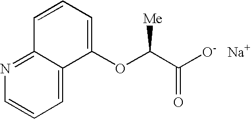

A solution of (S)-2-(quinolin-5-yloxy)-propionic acid methyl ester (Preparation 1) (1.30 g, 5.60 mmol) and 1 N sodium hydroxide solution (5.62 ml) in dioxane (40 ml) was heated at 60° C. for two hours. After this time tlc analysis indicated that starting material remained so additional 1N sodium hydroxide solution (1.4 ml) was added and heating at 60° C. continued for one hour. The reaction mixture was then concentrated under reduced pressure to afford the title compound as a yellow solid which was used without further purification.

LRMS (ESI+): m/z [M+H]⁺ 218.

### Preparation 3

### 1-[(2R)-4-Benzoyl-2-methyl-piperazin-1-yl]-2-bromo-propan-1-one

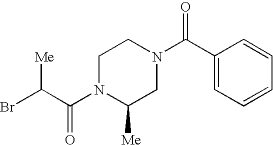

To a solution of (3R)-(3-methyl-piperazin-1-yl)-phenyl-methanone (J. Med. Chem. (2000), 43(23), 4499) (4.06 g, 19.6 mmol) in dichloromethane (60 ml) and N,N-dimethylformamide (2 ml) were added 2-bromopropionic acid (3.0 g, 19.6 mmol), O-(1H-benzotriazol-1-yl)-N,N,N′,N′-tetramethyluronium hexafluorophosphate (8.18 g, 21.6 mmol) and triethylamine (2.74 ml, 19.6 mmol). The reaction mixture was stirred at room temperature for 48 hours and then washed with water (50 ml), 10% w/v aqueous citric acid solution (50 ml) and saturated aqueous sodium hydrogencarbonate solution (50 ml). The solution was dried (MgSO₄) and solvent evaporated under reduced pressure. The crude product was purified by column chromatography on silica gel eluting with a gradient system of dichloromethane: methanol (99:1) changing to dichloromethane:methanol (95:5). The product was then purified further by column chromatography on silica gel eluting with a gradient system of ethyl acetate:dichloromethane (30:70) changing to ethyl acetate: dichloromethane (45:55) to afford the title compound as a white solid (3.80 g).

LRMS (ESI+): m/z [M+H]⁺ 340.

### Preparation 4

### 4-Hydroxy-3-methoxy-N-methyl-benzamide

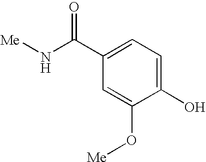

To a mechanically stirred suspension of vanillic acid (84 g, 0.5 mol) in dry THF (300 ml) was added portion-wise carbonyldiimidazole (97 g, 0.6 mol). Gaseous evolution was observed and the mixture became homogeneous after approx 5 minutes. After further stirring for 30 minutes an off-white precipitate form and stirring was maintained for a further 3 hours under nitrogen. After this time LCMS analysis indicated that the vanillic acid had been consumed and a solution of 2 M methylamine in THF (1000 ml, 2 mol) was added in one portion and the mixture stirred for a further 16 hours to generate a tan precipitate. The precipitate was filtered, washed with THF (2×250 ml) followed by ether (300 ml) and dried under vacuum to afford the N-methylammonium salt of the title compound (87 g, 82%).

LRMS (ESI+): m/z [M+H]⁺ 182.

A portion of the ammonium salt (1.06 g, 5 mmol) was refluxed in ethyl acetate under a stream of nitrogen for 2 hours. The reaction was evaporated to give an oily solid, which was triturated with ethyl acetate to afford the title compound as a tan solid (750 mg)

LRMS (ESI+): m/z [M+H]⁺ 182.

### Preparation 5

### (2S)-2-(2-Methoxy-4-methylcarbamoyl-phenoxy)-propionic acid

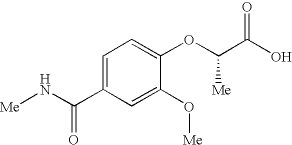

To a stirred mixture of triphenylphosphine (42 g, 0.16 mol), 4-hydroxy-3-methoxy-N-methyl-benzamide (Preparation 4) (20 g, 0.11 mol) and methyl (R)-lactate (12 g, 0.12 mol) in dry THF (300 ml) at −5° C. was added dropwise a solution of di-isopropyl azodicarboxylate (32 ml, 0.16 mmol) in dry THF (100 ml) over 30 minutes. The solution was allowed to warm to room temperature and stirred for 2 hours. After this time tlc analysis indicated that the starting phenol had been consumed. The crude reaction mixture was evaporated under reduced pressure to give a viscous oil. To a stirred solution of this oil in methanol (150 ml) was added lithium hydroxide monohydrate (5.4 g, 0.13 mol) and the mixture stirred for 48 hours. After this time LCMS analysis indicated that the intermediate methyl ester had been consumed. The crude reaction mixture was evaporated and partitioned between ether (500 ml) and water (150 ml). The aqueous phase was separated and washed with ether (2×300 ml) and ethyl acetate (2×300 ml). The aqueous phase was then acidified to pH 2 using concentrated aqueous hydrochloric acid resulting in the formation of a white precipitate. This precipitate was filtered off, washed with water (2×100 ml) and recrystallised from ethanol water (1:1 by volume, 80 ml per 20 g) to afford the title compound as a white solid (21 g, 75%).

LRMS (ESI+): m/z [M+H]⁺ 254.

### Preparation 6

### 3-Methoxy-4-[(1S)-1-methoxycarbonyl-ethoxy]-benzoic acid methyl ester

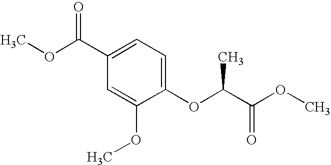

Diisopropyl azodicarboxylate (4 mL, 20 mmol) in tetrahydrofuran (15 mL) was added dropwise to an ice-cooled solution of methyl vanillate (3.64 g, 20 mmol), methyl (R)-lactate (2.08 g, 20 mmol) and triphenyl phosphine (5.24 g, 20 mmol) in tetrahydrofuran (30 mL) and the reaction mixture was stirred at room temperature for 18 hours. The solvent was then evaporated under reduced pressure and the residue was stirred in a mixture of diethyl ether (50 mL) and hexane (50 mL). The resulting precipitate was filtered off and the filtrate was concentrated in vacuo. Purification of the residue by column chromatography on silica gel, eluting with hexane:ethyl acetate, 85:15 to 75:25 afforded the title compound in 71% yield, 3.8 g

LRMS (ES⁺): m/z [M+H]⁺ 269

### Preparation 7

### 4-[(1S)-1-Carboxy-ethoxy]-3-methoxy-benzoic acid methyl ester

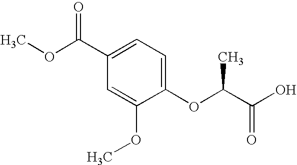

Lithium hydroxide monohydrate (4.2 g, 100 mmol) was added portionwise to a solution of the compound of Preparation 6 (32 g, 120 mmol) in methanol (150 mL) and the mixture was stirred at room temperature for 18 hours. The reaction mixture was then evaporated under reduced pressure and the residue was dissolved in water and washed with diethyl ether. The aqueous mixture was acidified with 2M hydrochloric acid to pH4 and was then extracted with ethyl acetate (3×150 mL). The combined organic extracts were dried over magnesium sulfate and concentrated in vacuo. The resulting foam was dried under reduced pressure to afford the title compound in 91% yield, 23 g.

LRMS (ES⁺): m/z [M+H]⁺ 255

### Preparation 8

### 4-{(1S)-2-[(2R)-4-Benzoyl-2-methyl-piperazin-1-yl]-1-methyl-2-oxo-ethoxy}-3-methoxy-benzoic acid methyl ester

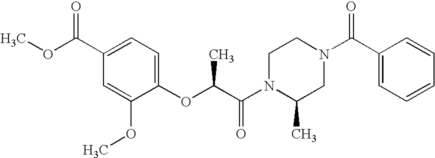

Hünig's base (1.8 mL, 10.5 mmol) was added to a solution of the compound of Preparation 7 (1.8 g, 7 mmol), (3R)-(3-methyl-piperazin-1-yl)-phenyl-methanone [(1.6 g, 7.7 mmol), J. Med. Chem. 43(23), 4499; 2000], and 3-(diethoxyphosphoryloxy)-1,2,3-benzotriazin-4(3H)-one (2.75 g, 9.1 mmol) in dichloromethane (16 mL). The mixture was stirred for 4 hours and was then evaporated under reduced pressure. The residue was taken up in ethyl acetate (200 mL) and washed with 10% sodium carbonate solution (2×50 mL) and brine (2×50 mL). The organic phase was dried over magnesium sulfate and concentrated in vacuo. Purification of the residue by column chromatography on silica gel, eluting with hexane:ethyl acetate, 25:75 to 5:95 afforded the title compound in 95% yield, 2.95 g.

LRMS (ES⁺): m/z [M+H]⁺ 441

### Preparation 9

### 4-{(1S)-2-[(2R)-4-Benzoyl-2-methylpiperazin-1-yl]-1-methyl-2-oxo-ethoxy}-3-methoxy-benzamide

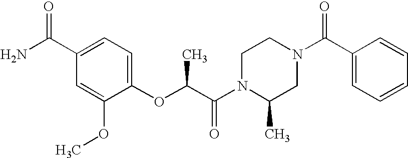

The compound of Preparation 8 (0.46 g, 1.04 mmol) and 2M ammonia in methanol (10 mL) were heated in a sealed vessel at 120° C. for 18 hours. The reaction mixture was then concentrated in vacuo and the residue was purified by column chromatography on silica gel, eluting with dichloromethane:methanol, 98:2 to 92:8, to afford the title compound as a colourless foam in 55% yield, 243 mg.

LRMS (ES⁺): m/z [M+H]⁺ 426

### Preparation 10

### 4-{(1S)-2-[(2R)-4-Benzoyl-2-methyl-piperazin-1-yl]-1-methyl-2-oxo-ethoxy}-N-dimethylaminomethylene-3-methoxy-benzamide

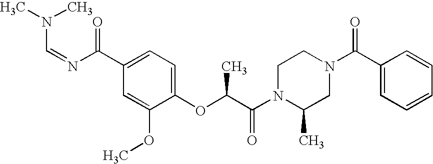

The compound of Preparation 9 (240 mg, 0.56 mmol) and N,N-dimethylformamide dimethylacetal (10 mL) were heated under reflux for 7 hours. The reaction mixture was then evaporated under reduced pressure to give a pale orange solid. The solid was purified by column chromatography on silica gel, eluting with dichloromethane:methanol, 99:1 to 97:3, to provide a colourless oil. The oil was then azeotroped with dichloromethane and the resulting foam was dried at 40° C. for 3 hours to afford the title compound in 94% yield, 255 mg.

LRMS (ES⁺): m/z [M+Na]⁺ 503

### Preparations 11 to 13

The following compounds, of the general formula shown below, were prepared by the method described above for Preparation 10 using the appropriate ketone and dimethylformamide dimethylacetal.

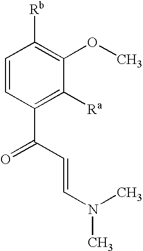

| No. | R^{a} | R^{b} | LRMS (ES⁺): m/z [M + H]⁺ |
|---|---|---|---|
| 11 | CH₃ | H | 220 |
| 12 | H | H | 206 |
| 13 | H | OCH₂—Ph | 312 |

Preparation 11 uses 1-(3-methoxy-2-methyl-phenyl)-ethanone, prepared as described in Tetrahedron 25(18), 4249; 1969.

### Preparation 14

### 3-(3-Methoxy-2-methyl-phenyl)-1-methyl-1H-pyrazole

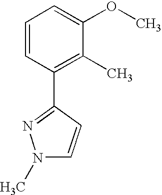

The compound of Preparation 11 (2.05 g, 9.35 mmol) was added portionwise to a solution of methylhydrazine (547 μL, 10.28 mmol) in acetic acid (15 mL) and the mixture was heated at 90° C. for 2.5 hours. The solvent was then evaporated under reduced pressure and the residue was dissolved in dichloromethane and washed with 10% sodium carbonate solution and saturated sodium hydrogen carbonate solution. The organic phase was dried over magnesium sulfate and concentrated in vacuo to give an orange oil. The oil was purified firstly by column chromatography on silica gel, eluting with dichloromethane:ethyl acetate, 99:1 to 97:3. This was followed by further purification using an Isolute® Flash silica column, eluting with dichloromethane:ethyl acetate, 99:1 to 97:3, to afford the title compound as a yellow oil in 31% yield, 568 mg.

LRMS (ES⁺): m/z [M+H]⁺ 203

### Preparations 15 to 16

The following compounds, of the general formula shown below, were prepared by the method described above for Preparation 14 using the appropriate enamine and hydrazine monohydrate.

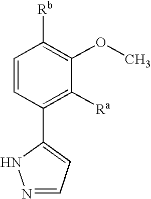

| No. | R^{a} | R^{b} | LRMS (ES⁺): m/z [M + H]⁺ |
|---|---|---|---|
| 15 | H | H | 175 |
| 16 | H | OCH₂Ph | 281 |

### Preparation 17

### 2-Methyl-3-(1-methyl-1H-pyrazol-3-yl)-phenol

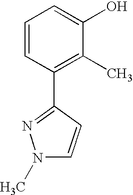

Boron tribromide (1M in dichloromethane, 8.16 mL, 8.16 mmol) was added dropwise to an ice-cooled solution of the compound of Preparation 14 (550 mg, 2.72 mmol) in dichloromethane (6 mL). The resulting solution was allowed to warm to room temperature and was stirred for 2 hours. The reaction mixture was then poured carefully onto ice and was neutralized with 10% sodium carbonate solution. The mixture was extracted with dichloromethane (2×50 mL) and the combined organic phases were dried over magnesium sulfate and concentrated in vacuo. The residue was chromatographed using an Isolute® Flash silica column, eluting with dichloromethane:methanol, 98:2 to 97:3, to give a brown oil. The oil was azeotroped with dichloromethane to afford the title compound as brown foam in 61% yield, 310 mg.

LRMS (ES⁺): m/z [M+H]⁺ 189

### Preparations 18 to 20

The following compounds, of the general formula shown below, were prepared by the method described above for Preparation 17 using the appropriate methoxybenzene derivative and boron tribromide.

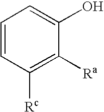

| No. | R^{a} | R^{c} | LRMS (ES⁺): m/z [M + H]⁺ |
|---|---|---|---|
| 18 | CH₃ | 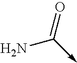 | 152 |
| 19 | CH₃ | 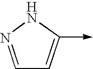 | 175 |
| 20 | H | 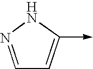 | 161 |

Preparation 18: title compound is prepared by dealkylation of 3-methoxy-2-methylbenzamide (J. Chem. Soc. Perkin Trans. 1, 2389-2396; 1984)

### Preparation 21

### (2S)-2-[3-(2H-Pyrazol-3-yl)-phenoxy]-propionic acid methyl ester

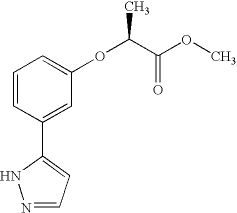

Diisopropyl azodicarboxylate (295 μL, 1.50 mmol) was added dropwise to an ice-cooled solution of the compound of Preparation 20 (240 mg, 1.5 mmol) methyl (R)-lactate (156 mg, 1.50 mmol) and triphenyl phosphine (393 mg, 1.5 mmol) in tetrahydrofuran (5 mL). The reaction mixture was stirred at room temperature for 18 hours and was then diluted with diethyl ether (50 mL) and washed with water (2×10 mL). The organic phase was dried over magnesium sulfate and concentrated in vacuo to give a yellow oil. The oil was purified on an Isolute® flash silica column, eluting with diethyl ether:dichloromethane, 80:20 to 95:5, to afford the title compound as a viscous yellow oil in 54% yield, 200 mg.

LRMS (ES⁻): m/z [M−H]⁻ 245

### Preparation 22

### (2S)-2-[2-Methyl-3-(2H-pyrazol-3-yl)-phenoxyl-propionic acid methyl ester

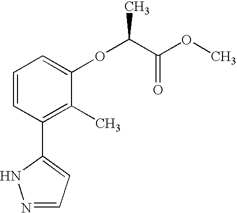

The title compound was prepared by the method described above for Preparation 21 using the compound of Preparation 19 and methyl (R)-lactate.

LRMS (ES⁻): m/z [M−H]⁻ 259

### Preparation 23

### (2S)-2-[3-(2H-Pyrazol-3-yl)-phenoxy]-propionic acid

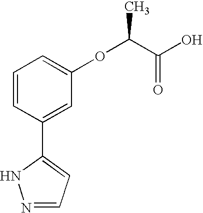

Lithium hydroxide monohydrate (80.9 mg, 1.93 mmol) was added portionwise to a solution of the compound of Preparation 21 (190 mg, 0.77 mmol) in methanol (6 mL) and the mixture was stirred at 60° C. for 8 hours. The reaction mixture was then evaporated under reduced pressure, and the residue was dissolved in water and washed with diethyl ether. The aqueous mixture, was acidified with 2M hydrochloric acid to pH4 and was then extracted with ethyl acetate (3×30 mL). The combined organic extracts were dried over magnesium sulfate and concentrated in vacuo. The resulting foam was dried under reduced pressure to afford the title compound in 75% yield, 135 mg.

LRMS (ES⁻): m/z [M−H]⁻ 231

### Preparation 24

### (2S)-2-[2-Methyl-3-(2H-pyrazol-3-yl)-phenoxy]-propionic acid

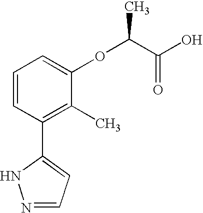

The title compound was prepared by the method described above for Preparation 22 using the compound of Preparation 22 and lithium hydroxide.

LRMS (ES⁻): m/z [M−H]⁻ 245

### Preparation 25

### 1-[(2R)-4-Benzoyl-2-methyl-piperazin-1-yl]-2-bromo-propan-1-one

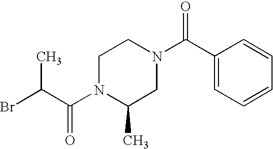

2-Bromopropionic acid (1.53 g, 10 mmol) was added to a solution of N,N′-dicyclohexylcarbodiimide (2.06 g, 10 mmol) in dichloromethane (40 mL), cooled to −15° C. The reaction mixture was stirred for 20 minutes before (2R)-(3-methyl-piperazin-1-yl)-phenyl-methanone [(2.04 g, 10 mmol), J. Med. Chem. 43(23), 4499; 2000] in dichloromethane (16 mL) was added dropwise. The reaction mixture was stirred at −15° C. for 90 minutes and was then diluted with diethyl ether (100 mL) and stirred for a further 10 minutes. The mixture was then filtered through Celite, washing through with diethyl ether and the filtrate was concentrated in vacuo. The residue was purified by column chromatography on silica gel, eluting with hexane:ethyl acetate, 40:60 to 20:80 to afford the title compound in 76% yield, 2.5 g.

LRMS (ES⁺): m/z [M+H]⁺ 340

### Preparation 26

### 3-{2-[(2R)-4-Benzoyl-2-methylpiperazin-1-yl]-1-methyl-2-oxo-ethoxy}-2-methyl-benzamide

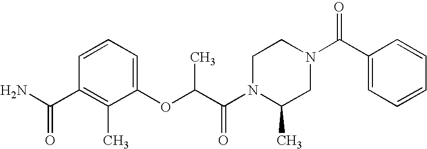

The compounds of Preparation 18 (180 mg, 1.19 mmol) and Preparation 25 (403.9 mg, 1.19 mmol) and cesium carbonate (387 mg, 1.19 mmol) in N,N-dimethylformamide (10 mL) were heated at 80° C. for 18 hours. The cooled reaction mixture was then evaporated under reduced pressure and the residue was partitioned between dichloromethane (50 mL) and water (10 mL). The organic phase was separated, dried over magnesium sulfate and concentrated in vacuo. The residue was purified on an Isolute® flash silica column, eluting with dichloromethane:methanol, 99:1 to 96:4, to give a colourless foam. The foam was azeotroped with dichloromethane and dried under reduced pressure to afford the title compound in 98% yield, 475 mg.

LRMS (ES⁺): m/z [M+H]⁺ 410

### Preparation 27

### 1-[(2R)-4-Benzoyl-2-methyl-piperazin-1-yl]-2-(3-methoxy-5-nitro-phenoxy)-propan-1-one

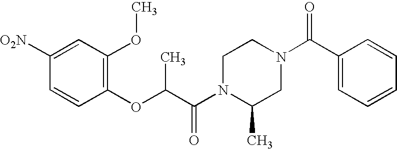

The title compound was prepared by the method described above for Preparation 26 using the compound of Preparation 25 and 2-methoxy-5-nitrophenol.

LRMS (APCI⁺): m/z [M+H]⁺ 428

### Preparation 28

### 3-{2-[(2R)-4-Benzoyl-2-methylpiperazin-1-yl]-1-methyl-2-oxoethoxy}-N-(dimethylamino)methylene]-2-methylbenzamide

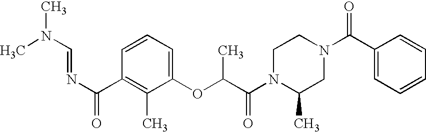

The title compound was prepared by the method described above for Preparation 10 using the compound of Preparation 26 and N,N-dimethylformamide dimethylacetal.

LRMS (APCI⁺): m/z [M+H]⁺ 465

### Preparation 29

### 3-{2-[(2R)-4-Benzoyl-2-methyl-piperazin-1-yl]-1-methyl-2-oxo-ethoxy}-N-hydroxyaminomethylene-2-methyl-benzamide

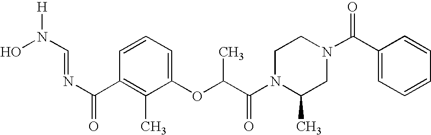

Sodium hydroxide solution (1M, 1.06 mL, 1.06 mmol) was added dropwise to a suspension of hydroxylamine hydrochloride (73.6 mg, 1.06 mmol) in acetic acid (6 mL). The compound of Preparation 28 (410 mg, 0.88 mmol) was then added portionwise and the resulting solution was stirred at room temperature for 30 minutes. The solvent was evaporated under reduced pressure and the oily residue was partitioned between dichloromethane (30 mL) and water (6 mL), and neutralized with 10% sodium carbonate solution. The organic phase was separated and the aqueous phase was re-extracted with dichloromethane (30 mL). The combined organic layers were dried over magnesium sulfate and concentrated in vacuo. The residue was purified on an Isolute® flash silica column, eluting with dichloromethane:methanol, 99:1 to 96:4, to afford the title compound as a colourless foam in 78% yield, 310 mg.

LRMS (ES⁻): m/z [M−H]⁻ 451

### Preparation 30

### 2-(4-Amino-2-methoxy-phenoxy)-1-[(2R)-4-benzoyl-2-methyl-piperazin-1-yl]-propan-1-one

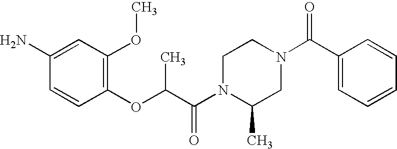

25% Pd/C (150 mg) and ammonium formate (443 mg, 7 mmol) were added to a solution of the compound of Preparation 27 (600 mg, 1.4 mmol) in ethanol (30 mL) and the mixture was heated at 70° C. for 2 hours. The reaction mixture was then cooled and filtered through Arbocel®, washing through with ethanol (10 mL). The filtrate was concentrated in vacuo and the residue was purified by column chromatography on silica gel, eluting with dichloromethane:methanol, 95:5, to afford the title compound as a pale pink solid in 80% yield, 443 mg.

LRMS (APCI⁺): m/z [M+H]⁺ 398

### Preparation 31

### 4-Benzyloxy-3-methoxy-benzamidine

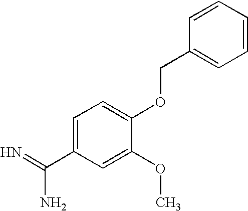

ⁿButyl lithium (1.6M in hexane, 35.9 mL, 57.47 mmol) was added dropwise to a solution of hexamethyldisilazane (11.64 mL, 55.17 mmol) in diethyl ether (60 mL), cooled to −5° C., and the reaction mixture was stirred for 30 minutes at a constant temperature of 0° C. 3-Methoxy-4-(phenylmethoxy)-benzonitrile (5.5 g, 22.99 mmol) was added portionwise and the mixture was stirred at room temperature 4 hours. The reaction mixture was then poured onto ice cold 2M hydrochloric acid (100 mL) and was washed with diethyl ether (2×100 mL). The aqueous phase was basified to pH10 with 6M sodium hydroxide solution and was extracted with dichloromethane (3×100 mL). The combined organic extracts were dried over magnesium sulfate and concentrated in vacuo to afford the title compound as a white solid in 68% yield, 3.99 g.

LRMS (ES⁺): m/z [M+H]⁺ 257

### Preparation 32

### 2-(4-Benzyloxy-3-methoxy-phenyl)-1H-imidazole

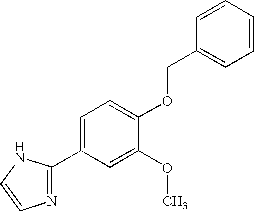

The compound of Preparation 31 (1 g, 3.90 mmol) in chloroform (40 mL) was added to a solution of chloroacetaldehyde (336 mg, 3.90 mmol) in chloroform (10 mL) and the mixture was stirred at room temperature for 18 hours. The resulting white precipitate was filtered off and the filtrate was washed with saturated sodium hydrogen carbonate solution (30 mL). The aqueous phase was re-extracted with 10% methanol in dichloromethane (2×50 mL) and the combined organic extracts were dried over magnesium sulfate and concentrated in vacuo. The residue was purified on an Isolute® flash silica column, eluting with dichloromethane:methanol, 98:2 to 96:4, to give a brown foam. The foam was purified further on an Isolute® flash silica column, eluting with dichloromethane:methanol, 99.5:0.5 to 98:2, to give a beige foam. The foam was then azeotroped with dichloromethane and dried at 40° C. to afford the title compound in 16% yield, 180 mg.

LRMS (ES⁺): m/z [M+H]⁺ 281

### Preparation 33

### 4-(1H-imidazol-2-yl)-2-methoxy-phenol

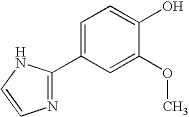

To a solution of the compound of Preparation 32 (170 mg, 0.61 mmol) in methanol (10 mL) was added portionwise 10% Pd/C (85 mg) and ammonium formate (191 mg, 3.03 mmol). The mixture was heated under reflux for 2 hours. The reaction mixture was then cooled and filtered through Arbocel®, washing through with a mixture of dichloromethane and methanol (50:50, 250 mL). The filtrate was concentrated in vacuo and the residue was purified on an Isolute® flash silica column, eluting with dichloromethane:methanol, 99:1 to 96:4, to afford the title compound as a white solid in 91% yield, 105 mg.

LRMS (ES⁺): m/z [M+H]⁺ 191

### Preparation 34

### 2-Methoxy-4-(2H-pyrazol-3-yl)-phenol

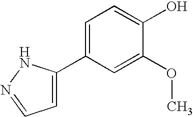

The title compound was prepared by the method described above for Preparation 33 using the compound of Preparation 16.

LRMS (ES⁺): m/z [M+H]⁺ 191

### Preparation 35

### 1-Benzyloxy-4-bromo-2-methoxy-benzene

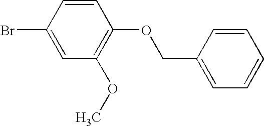

A mixture of 4-bromo-2-methoxyphenol (5 g, 24.63 mmol), benzyl bromide (4.21 g, 24.63 mmol) and cesium carbonate (8 g, 24.63 mmol) in acetone (60 mL) were heated under reflux for 4 hours. The cooled reaction mixture was then evaporated under reduced pressure and the residue was partitioned between diethyl ether (200 mL) and water (80 mL). The aqueous phase was separated and re-extracted with diethyl ether (200 mL). The combined organic extracts were dried over magnesium sulfate and concentrated in vacuo. The residue was azeotroped with dichloromethane to afford the title compound as white solid in 100% yield, 7.19 g.

LRMS (ES⁺): m/z [M+Na]⁺ 317

### Preparation 36

### 1-(4-Benzyloxy-3-methoxy-phenyl)-1H-pyrazole

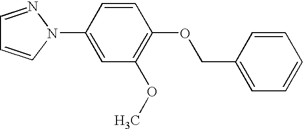

A mixture of the compound of Preparation 35 (1.5 g, 5.12 mmol), pyrazole (697 mg, 10.23 mmol), copper (I) iodide (195 mg, 1.02 mmol) and potassium carbonate in N,N-dimethylformamide (10 mL) was heated at 140° C. for 18 hours. The cooled reaction mixture was then evaporated under reduced pressure and the residue was partitioned between dichloromethane (50 mL) and water (30 mL). The aqueous phase was separated and re-extracted with diethyl ether (200 mL). The combined organic extracts were dried over magnesium sulfate and concentrated in vacuo. The residue was purified by column chromatography on silica gel, eluting with dichloromethane:methanol, 100:0 to 96:4, to afford the title compound as a white solid in 82% yield, 1.18 g.

LRMS (ES⁺): m/z [M+H]⁺ 281

### Preparation 37

### 2-Methoxy-4-pyrazol-1-yl-phenol

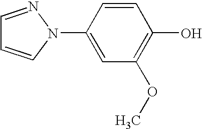

The title compound was prepared by the method described above for Preparation 33 using the compound of Preparation 36.

LRMS (ES⁺): m/z [M+H]⁺ 191

### Preparation 38

### (2S)-2-(Quinolin-5-yloxy)-propionic acid methyl ester

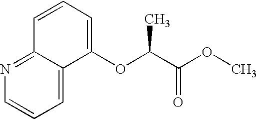

Triphenylphosphine (2.17 g, 8.27 mmol) was added to a stirred suspension of diisopropyl azodicarboxylate (1.47 mL, 7.56 mmol) in tetrahydrofuran (25 mL) at −5° C. 5-Hydroxyquinoline (1.0 g, 6.90 mmol) and methyl-(R)-lactate (0.66 mL, 6.90 mmol) were added and the solution was allowed to stir at room temperature for 14 hours. The reaction mixture was then diluted with ethyl acetate (50 mL), washed with water (30 mL), saturated potassium carbonate solution (30 mL) and brine (30 mL). The solution was dried over magnesium sulfate and concentrated in vacuo. The residue was purified by column chromatography on silica gel, eluting with pentane:ethyl acetate, 80:20 to 60:40, to afford the title compound as a white solid in 81% yield, 1.30 g.

LRMS (ES⁺): m/z [M+H]⁺ 232

### Preparation 39

### (2S)-2-(Isoquinolin-5-yloxy)-propionic acid methyl ester

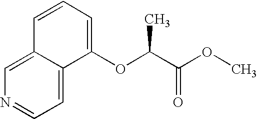

The title compound was prepared by the method described above for Preparation 38 using 5-hydroxyisoquinoline and methyl-(R)-lactate.

LRMS (APCI⁺): m/z [M+H]⁺ 232

### Preparation 40

### (2S)-2-(Quinolin-5-yloxy)-propionic acid sodium salt

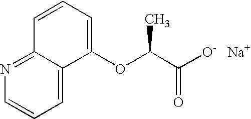

A solution of the compound of Preparation 38 (1.30 g, 5.60 mmol) and 1M sodium hydroxide solution (5.62 mL) in 1,4-dioxane (40 mL) was heated at 60° C. for two hours. After this time tlc analysis indicated that starting material remained so additional 1M sodium hydroxide solution (1.4 ml) was added and heating continued at 60° C. for one hour. The reaction mixture was then concentrated in vacuo to yield the title compound as a yellow solid in quantitative yield.

LRMS (ES⁺): m/z [M+H]⁺ 218

### Preparation 41

### (2S)-2-(Isoquinolin-5-yloxy)-propionic acid sodium salt

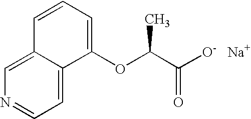

The title compound was prepared by the method described above for Preparation 40 using the compound of Preparation 39.

LRMS (APCI⁺): m/z [M+H]⁺ 218

### Preparation 42

### (2S)-1-[(2R)-4-Benzoyl-2-methyl-piperazin-1-yl]-2-(quinolin-5-yloxy)-propan-1-one

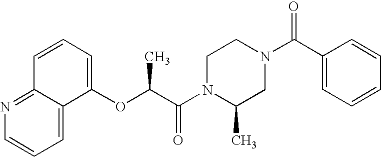

A solution of the compound of Preparation 40 (1.0 g, 4.6 mmol), (3R)-(3-methyl-piperazin-1-yl)-phenyl-methanone [(0.94 g, 4.6 mmol) J. Med. Chem., 43(23), 4499; 2000], O-(1H-benzotriazol-1-yl)-N,N,N′,N′-tetramethyluronium hexafluorophosphate (2.62 g, 6.9 mmol) and triethylamine (1.93 ml, 13.8 mmol) in N,N-dimethylformamide (30 ml) was stirred at room temperature for 14 hours. The reaction mixture was then diluted with dichloromethane (100 ml), washed with water (3×50 ml), dried over magnesium sulfate and concentrated in vacuo. The residue was purified by column chromatography on silica gel eluting with dichloromethane:methanol, 98:2, to afford the title compound as a white solid in 49% yield, 0.919 g.

LRMS (ES⁺): m/z [M+H]⁺ 404

### Preparation 43

### (2S)-1-[(2R)-4-Benzoyl-2-methyl-piperazin-1-yl)-2-(isoquinolin-5-yloxy)-propan-1-one

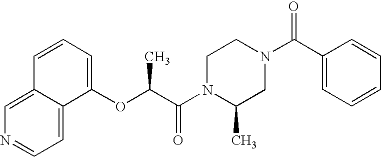

The title compound was prepared by the method described above for Preparation 42 using the compound of Preparation 41 and (3R)-(3-methyl-piperazin-1-yl)-phenyl-methanone (J. Med. Chem., 43(23), 4499; 2000.

LRMS (APCI⁺): m/z [M+H]⁺ 404

### Preparation 44

### (2S)-1-[(2R)-4-Benzoyl-2-methyl-piperazin-1-yl]-2-(8-bromo-quinolin-5-yloxy)-propan-1-one

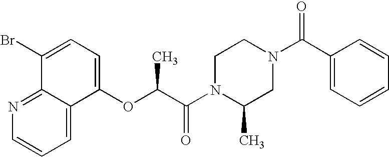

A mixture of the compound of Preparation 42 (450 mg, 1.1 mmol) and N-bromosuccinimide (795 mg, 4.4 mmol) in acetonitrile (90 mL) was stirred at room temperature for 2 hours. The solvent was then evaporated under reduced pressure and the residue was dissolved in ethyl acetate (50 mL) and washed with water (2×20 mL). The organic phase was dried over magnesium sulfate and concentrated in vacuo. The residue was purified by column chromatography on silica gel, eluting with ethyl acetate:pentane, 33:67 to 100:0 to afford the title compound as a white solid in 98% yield, 526 mg.

LRMS (ES⁺): m/z [M+H]⁺ 482/484

### Preparation 45

### (2S)-1-[(2R)-4-Benzoyl-2-methyl-piperazin-1-yl]-2-{8-[2-(2-trimethylsilanyl-ethoxymethyl)-2H-pyrazol-3-yl]-quinolin-5-yloxy}-propan-1-one

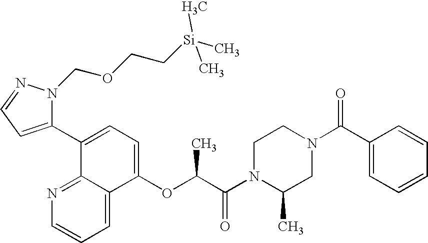

Tetrakis(triphenylphosphine)palladium(0)(6 mg, cat), 1-[[(2-(trimethylsilyl)ethoxy]methyl]pyrazolyl-5-boronic acid [(50 mg, 0.1 mmol), J. Med. Chem. 41 2019-2028; 1998] and sodium carbonate (175 mg, 1.6 mmol) in water (0.5 mL) were added to the compound of Preparation 44 (50 mg, 0.1 mmol) in tetrahydrofuran (2 mL) and the mixture was heated under reflux for 18 hours. The reaction mixture was then evaporated under reduced pressure and the residue was dissolved in dichloromethane and washed with water (5 mL) and brine (2×5 mL). The organic phase was dried over magnesium sulfate and concentrated in vacuo to give an orange oil. The oil was purified by column chromatography on silica gel, eluting with dichloromethane:methanol, 99:1 to 96:4 to afford the title compound as a yellow solid.

LRMS (APCI⁺): m/z [M+H]⁺ 600

### Preparation 46

### (2S)-1-[(2R)-4-Benzoyl-2-methyl-piperazin-1-yl]-2-(1-oxy-quinolin-5-yloxy)-propan-1-one

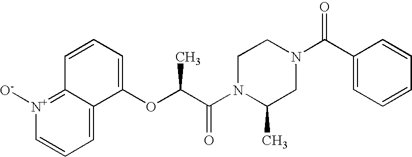

meta-Chloroperbenzoic acid (1.54 g, 4.44 mmol) was added to an ice-cooled solution of the compound of Preparation 42 (1.5 g, 3.7 mmol) in dichloromethane (15 mL) and the mixture was stirred at room temperature for 3 hours. The reaction mixture was then diluted with dichloromethane (10 mL) and washed with saturated aqueous potassium carbonate solution (10 mL) and water (2×10 mL). The organic layer was dried over magnesium sulfate and concentrated in vacuo to afford the title compound as a white solid in quantitative yield, 1.8 g.

LRMS (APCI⁺): m/z [M+H]⁺ 420

### Preparation 47

### 5-{2-[(2R)-4-Benzoyl-2-methyl-piperazin-1-yl]-1-methyl-2-oxo-ethoxy}-quinoline-2-carboxylic acid methyl ester

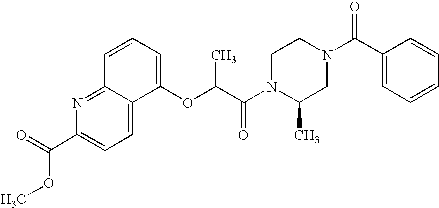

The chloro compound of Preparation 66 (130 mg, 0.3 mmol), dichloro-bis(triphenylphosphine)palladium (24 mg, 0.03 mmol) and triethylamine (83 μL, 0.6 mmol) were dissolved in a mixture of methanol (3 mL) and N,N-dimethylformamide (0.5 mL), and transferred to a sealed vessel. The vessel was heated to 100° C. and the mixture was stirred under 100 psi of carbon monoxide gas for 42 hours. The reaction mixture was then filtered through Arbocel®, washing through with methanol and the filtrate was concentrated in vacuo. Purification of the residue by column chromatography on silica gel, eluting with ethyl acetate:pentane, 75:25 to 100:0, afforded the title compound in 41% yield, 50 mg.

LRMS (APCI⁺): m/z [M+H]⁺ 462

### Preparation 48

### 5-{2-[(2R)-4-Benzoyl-2-methyl-piperazin-1-yl]-1-methyl-2-oxo-ethoxy}-quinoline-2-carboxylic acid

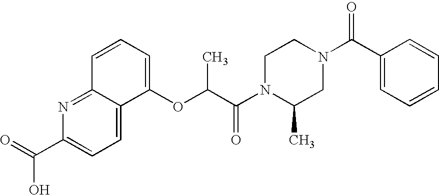

1M Sodium hydroxide solution (130 μL, 0.135 mmol) was added to a solution of the compound of Preparation 47 (40 mg, 0.09 mmol) in dioxane (3 mL) and the mixture was heated at 60° C. for 3 hours. The solvent was then evaporated under reduced pressure and the residue was dissolved in water (20 mL) and washed with ethyl acetate. The phases were separated and the organic phase was discarded. The aqueous phase was acidified with glacial acetic acid and re-extracted with ethyl acetate. The retained organic phase (2^{nd} washing) was dried over magnesium sulfate and concentrated in vacuo to afford the title compound as a pale yellow solid in 67% yield, 26 mg.

LRMS (APCI⁺): m/z [M+H]⁺ 448

### Preparation 49

### (2S)-1-[(2R)-4-Benzoyl-2-methyl-piperazin-1-yl)-2-(2-benzylamino-quinolin-5-yloxy)-propan-1-one

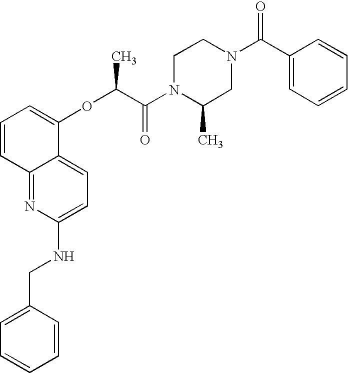

A mixture of the compound of Example 60 (50 mg, 0.11 mmol), tetrabutyl ammonium fluoride (30 mg, 0.22 mmol), triethylamine (0.16 mL, 1.1 mmol) and benzylamine (0.13 mL, 1.1 mmol) in dimethylsulfoxide (1 mL) was heated at 120° C. for 72 hours. The reaction mixture was then purified directly by HPLC using a Phenomenex Luna C₁₈ system, eluting with water/acetonitrile/trifluoroacetic acid (5:95:0.1):acetonitrile, 95:5 to 5:95,, to afford the title compound in 34% yield.

LRMS (APCI⁺): m/z [M+H]⁺ 509

### Preparation 50

### (3R)-3-Methyl-4-[(2S)-2-(quinolin-5-yloxy)-propionyl]-piperazine-1-carboxylic acid tert-butyl ester

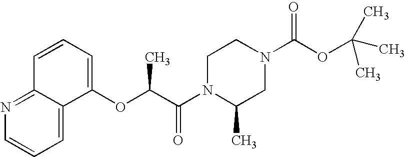

1-Hydroxybenzotriazole hydrate (249 mg, 1.87 mmol) was added to a suspension of the compound of Preparation 40 (400 mg, 1.7 mmol) in N,N-dimethylformamide to form a solution. 1-Ethyl-3-(3-dimethyl amino propyl) carbodiimide (351 mg, 1.87 mmol) and triethylamine (0.7 mL, 5.1 mmol) were added and the solution was stirred for 5 minutes. (3S)-3-Methyl-piperazine-1-carboxylic acid tert-butyl ester (335 mg, 1.7 mmol) was added and the mixture was stirred for 18 hours. The solvent was then evaporated under reduced pressure and the residue was dissolved in ethyl acetate (30 mL) and washed with water (20 mL). The organic phase was dried over magnesium sulfate and concentrated in vacuo to afford the title compound in 48% yield, 332 mg.

LRMS (APCI⁺): m/z [M+H]⁺ 400

### Preparation 51

### (2S)-1[(2R)-2-Methyl-piperazin-1-yl]-2-(quinolin-5-yloxy)-propan-1-one; hydrochloride

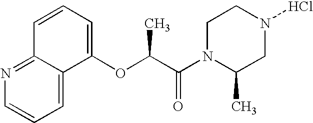

The compound of Preparation 50 (300 mg, 0.75 mmol) was stirred in 4M hydrochloric acid in dioxane (3 mL) for 2 hours. The reaction mixture was concentrated in vacuo to afford the title compound as a pale yellow solid in quantitative yield, 348 mg.

LRMS (APCI⁺): m/z [M+H]⁺ 300

### Preparation 52

### (2S)-1-[(2R)-4-Benzoyl-2-methyl-piperazin-1-yl]-2-(2.8-dichloro-quinolin-5-yloxy)-propan-1-one

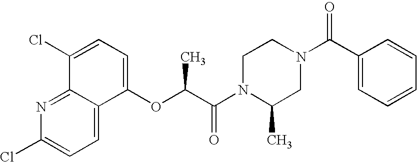

A mixture of the compound of Example 60 (200 mg, 0.4 mmol) and N-chlorosuccinimide (61 mg, 0.4 mmol) in acetonitrile (10 mL) was heated at 40° C. for 48 hours. The solvent was then evaporated under reduced pressure and the residue was dissolved in dichloromethane (20 mL) and washed with water (2×10 mL). The organic phase was dried over magnesium sulfate and concentrated in vacuo to give a yellow oil. The residue was purified by column chromatography on silica gel, eluting with dichloromethane:methanol, 98:2, to afford the title compound as a white solid in 78% yield, 169 mg.

LRMS (APCI⁺): m/z [M+H]⁺ 472

### Preparation 53

### (2S)-1-[(28)-2-Methyl-4-(pyridine-2-carbonyl)-piperazin-1-yl]-2-(quinolin-5-yloxy)-propan-1-one

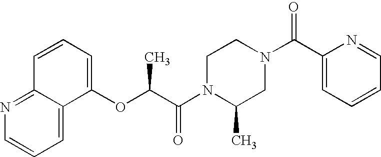

A mixture of the compound of Preparation 51 (150 mg, 0.5 mmol), picolinic acid (49 mg, 0.6 mmol) O-(1H-benzotriazol-1-yl)-N,N,N′,N′-tetramethyluronium hexafluorophosphate (190 mg, 0.75 mmol) and triethylamine (0.93 mL, 10 mmol) in dichloromethane (3 mL) was stirred for 18 hours at room temperature. The reaction mixture was then diluted with further dichloromethane and washed with sodium hydroxide (10 mL) and water (2×10 mL). The organic phase was dried over magnesium sulfate and concentrated in vacuo. Purification of the residue by column chromatography on silica gel, eluting with dichloromethane:methanol, 99:1 to 95:5, afforded the title compound as a white solid in 49% yield, 99 mg.

LRMS (APCI⁺): m/z [M+H]⁺ 405

### Preparation 54

### (2S)-1-4-(2-Fluoro-benzoyl)-(2R)-2-methyl-piperazin-1-yl]-2-(quinolin-5-yloxy)-propan-1-one

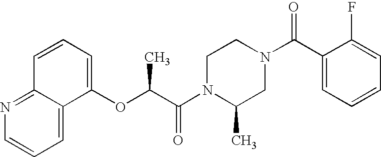

A mixture of the compound of Preparation 51 (150 mg, 0.5 mmol), 2-fluorobenzoyl chloride and triethylamine in dichloromethane (3 mL) was stirred at room temperature for 18 hours. The reaction mixture was then diluted with dichloromethane and washed with water (2×10 mL). The organic phase was dried over magnesium sulfate and concentrated in vacuo. Purification by column chromatography on silica gel, eluting with dichloromethane:methanol, 98:2, afforded the title compound as a white solid in 73% yield, 153 mg.

LRMS (APCI⁺): m/z [M+H]⁺ 422

### Preparation 55

### 4-Bromo-2-trifluoromethoxy-phenol

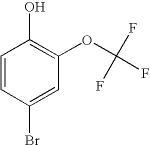

Bromine (449 mg, 2.81 mmol) was added to a solution of 2-(trifluoromethoxy)phenol (500 mg, 2.81 mmol) and sodium acetate (169 mg, 2.81 mmol) in acetic acid (5 mL) and the solution was stirred for 1 hour. The reaction mixture was then diluted with water (30 mL) and extracted with ethyl acetate (2×50 mL). The organic extracts were combined, dried over magnesium sulfate and concentrated in vacuo to give a colourless oil. The oil was purified by column chromatography on silica gel, eluting with pentane:ethyl acetate, 85:15, to afford the title compound as a white solid in 23% yield.

LRMS (APCI⁺): m/z [M+H]⁺ 255/257

### Preparation 56

### (2S)-2-(4-Bromo-2-trifluoromethoxy-phenoxy)-propionic acid methyl ester

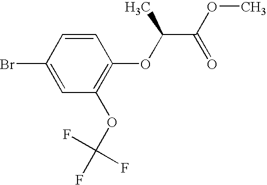

Diisopropyl azodicarboxylate (414 μL, 2.14 mmol) was added dropwise to an ice-cooled solution of the compound of Preparation 55 (500 mg, 1.95 mmol) methyl (R)-lactate.(202 mg, 1.95 mmol) and triphenyl phosphine (614 mg, 2.34 mmol) in tetrahydrofuran (20 mL). The reaction mixture was stirred at room temperature for 18 hours and was then evaporated under reduced pressure. The residue was dissolved in ethyl acetate and washed with 10% potassium carbonate solution (2×10 mL). The organic phase was dried over magnesium sulfate and concentrated in vacuo. The residue was purified by column chromatography on silica gel, eluting with dichloromethane:methanol, 95:5, to afford the title compound as a colourless oil in 76% yield, 638 mg.

LRMS (APCI⁺): m/z [M+H]⁺ 305/307

### Preparation 57

### (2S)-2-(4-Bromo-2-trifluoromethoxy-phenoxy)-propionic acid

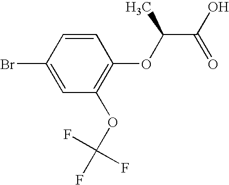

A mixture of the compound of Preparation 56 (574 mg, 1.67 mmol) and 1M sodium hydroxide solution (4.2 mL, 4.2 mmol) in dioxane (10 mL) was stirred at 55° C. for 3 hours. The solvent was then evaporated under reduced pressure and the residue was dissolved in water (30 mL). The aqueous solution was acidified to pH2 with 2M hydrochloric acid and extracted with ethyl acetate (2×50 mL). The combined organic extracts were dried over magnesium sulfate and concentrated in vacuo to afford the title compound as a yellow solid in 80% yield.

LRMS (APCI⁺): m/z [M+H]⁺ 327

### Preparation 58

### (2S)-1-[(2R)-4-Benzoyl-2-methyl-piperazin-1-yl]-2-(4-bromo-2-trifluoromethoxy-phenoxy)-propan-1-one

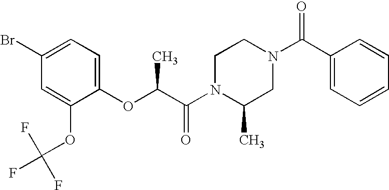

A solution of the compound of Preparation 57 (150 mg, 0.46 mmol), (3R)-(3-methyl-piperazin-1-yl)-phenyl-methanone [(93 mg, 0.46 mmol) J. Med. Chem., 43(23), 4499; 2000], O-(1H-benzotriazol-1-yl)-N,N,N′,N′-tetramethyluronium hexafluorophosphate (260 mg, 0.69 mmol) and triethylamine (0.13 ml, 0.92 mmol) in dichloromethane (30 ml) was stirred at room temperature for 18 hours. The reaction mixture was then diluted with dichloromethane (50 ml) and washed with water (50 ml). The aqueous phase was re-extracted with dichloromethane (50 mL) and the combined organic extracts were dried over magnesium sulfate and concentrated in vacuo.

The residue was purified on an Isolute® flash silica column, eluting with dichloromethane:methanol, 99:1, to afford the title compound as a white foam in 90% yield, 213 mg.

LRMS (APCI⁺): m/z [M+H]⁺ 537/539

### Preparation 59

### 4-{(1S)-2-[(2R)-4-Benzoyl-2-methyl-piperazin-1-yl]-1-methyl-2-oxo-ethoxy-}-3-trifluoromethoxy-benzoic acid methyl ester

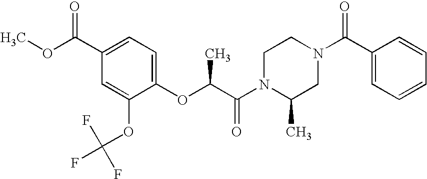

The compound of Preparation 58 (190 mg, 0.37 mmol), dichloro-bis(triphenylphosphine)palladium (30 mg, 0.04 mmol) and triethylamine (0.10 mL, 0.74 mmol) were dissolved in methanol (10 mL) and transferred to a sealed vessel. The vessel was heated to 100° C. and the mixture was stirred under 100 psi of carbon monoxide gas for 42 hours. The reaction mixture was then filtered through Arbocel®, washing through with methanol and the filtrate was concentrated in vacuo. The residue was purified on an Isolute® flash silica column, eluting with dichloromethane:methanol, 97:3 to afford the title compound as an orange solid in 85% yield, 155 mg.

LRMS (APCI⁺): m/z [M+H]⁺ 495

### Preparations 60 to 61

The following compounds, of the general formula shown below, were prepared by the method described above for Preparation 26 using the compound of Preparation 25 and the appropriate hydroxyquinoline or hydroxyisoquinoline.

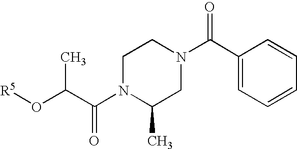

| No. | R⁵ | LRMS (APCl⁺): m/z [M + H]⁺ |
|---|---|---|
| 60 | 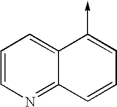 | 404 |
| 61 | 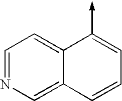 | 404 |

The following compounds, of the general formula shown below, were prepared by the method described above for Preparation 46 using the compounds of Preparations 60 and 61 and meta-chloroperbenzoic acid

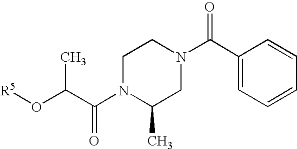

| No. | R⁵ | LRMS (APCl⁺): m/z [M + H]⁺ |
|---|---|---|
| 62 | 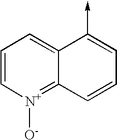 | 420 |
| 63 | 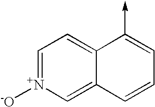 | 420 |

### Preparation 64

### (2S)-1-[(2R)-4-Benzoyl-2-methyl-piperazin-1-yl]-2-(1-oxy-isoquinolin-5-yloxy)-propan-1-one

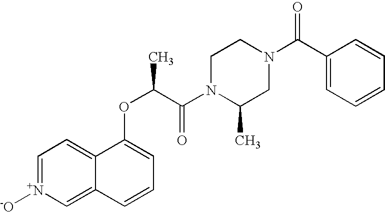

The title compound was prepared by the method described above for Preparation 46 using the compound of Preparation 43.

LRMS (APCI⁺): m/z [M+H]⁺ 420

### Preparation 65

### (2S)-1-[(2R)-4-Benzoyl-2-methyl-piperazin-1-yl]-2-(1-chloro-isoquinolin-5-yloxy)-propan-1-one

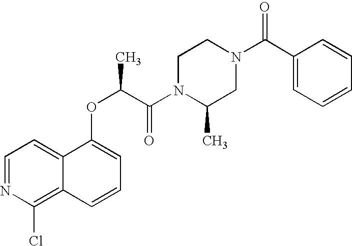

Phosphorus oxychloride (0.20 mL, 2.18 mmol) was added to a solution of the compound of Preparation 64 (305 mg, 0.73 mmol) in dichloromethane (15 mL) and the reaction mixture was heated at 100° C. for two hours. The mixture was then cooled to room temperature and poured onto water (75 mL). The resulting aqueous mixture was basified with concentrated ammonia solution and extracted with dichloromethane (2×75 mL). The combined organic extracts were then dried over magnesium sulfate and concentrated in vacuo. Purification of the residue by column chromatography on silica gel, eluting with dichloromethane:methanol, 99:1, afforded the title compound as a white foam in 40% yield, 127 mg.

LRMS (APCI⁺): m/z [M+H]⁺ 438

### Preparation 66

### 1-[(2R)-4-Benzoyl-2-methyl-piperazin-1-yl]-2-(2-chloro-quinolin-5-yloxy)-propan-1-one

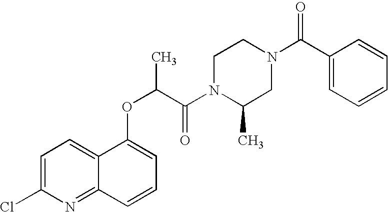

The title compound was prepared by the method described above for Preparation 65 using the compound of Preparation 62.

LRMS (APCI⁺): m/z [M+H]⁺ 438

### Preparation 67

### 2-Methoxy-4-methylsulfanyl-phenol

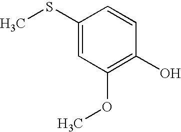

^{t}Butyl lithium (1.7M in pentane, 14.5 mL, 24.6 mmol) was added dropwise to a solution of 4-bromo-2-methoxyphenol (2.00 g, 9.85 mmol) in tetrahydrofuran (25 mL) cooled to −78° C. The mixture was stirred for 15 minutes and was then warmed to −40° C. and stirred for a further 30 minutes. Dimethyl disulfide (1.06 mL, 11.8 mmol) was added and the mixture was stirred at room temperature for 18 hours. Water was then added to the reaction and the resulting mixture was acidified to pH1 with 2M hydrochloric acid. The aqueous phase was separated and re-extracted with ethyl acetate. The combined organic fractions were then dried over magnesium sulfate and concentrated in vacuo to give an orange oil. This oil was purified by column chromatography on silica gel, eluting with pentane:diethyl ether, 95:5 to 80:20, to afford the title compound as a white solid in 48% yield, 800 mg.

¹HNMR(CDCl₃, 400 MHz) δ: 2.40(s, 3H), 3.90(s, 3H), 5.50(brs, 1H), 6.80-6.90(m, 3H)

### Preparation 68

### 6-Hydroxy-7-methoxy-3,4-dihydro-2H-isoquinolin-1-one

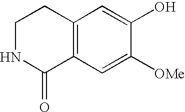

A mixture of lithium iodide (0.32 g, 2.41 mmol) and 6,7-dimethoxy-3,4-dihydro-2H-isoquinolin-1-one (0.5 g, 2.41 mmol) in 2,4,6-collidine (10 ml) was heated at 130° C. for 18 hours. The solvent was then evaporated under reduced pressure and the residue was purified by column chromatography on silica gel, eluting with dichloromethane:methanol, 96:4, to afford the title compound as a pale yellow solid, 0.19 g

LRMS (APCI⁺): m/z [M+H]⁺ 194

### Preparation 69

### Acetic acid 3-acetoxy-5-methyl-phenyl ester

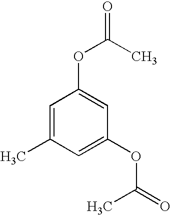

Acetic anhydride (22.6 mL, 0.24 mol) was added to an ice-cooled solution of 3,5-dihydroxytoluene (9.93 g, 0.08 mol) and triethylamine (56 mL, 0.40 mol) in dichloromethane (86 mL). The reaction mixture was warmed to room temperature and was stirred for 60 hours. Water (100 mL) was then added and the mixture was stirred vigorously for 3 hours. The organic layer was separated and the aqueous layer was re-extracted with dichloromethane (3×80 mL). The combined organic extracts were washed with brine (80 mL), dried over magnesium sulfate and concentrated in vacuo. The residue was purified by column chromatography on silica gel, eluting with ethyl acetate:pentane, 66:33, to afford the title compound as a colourless oil in 94% yield, 15.7 g.

LRMS (APCI⁺): m/z [M+NH₄]⁺ 226

### Preparation 70

### 1-(2,6-Dihydroxy-4-methyl-phenyl)-ethanone

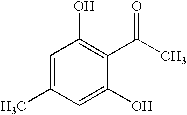

A solution of the compound of Preparation 69 (10.12 g, 48.6 mmol) in chlorobenzene (10 mL) was added dropwise to a suspension of aluminium chloride (19.44 g, 145.8 mmol) in chlorobenzene (50 mL), warmed to 90° C., and the mixture was stirred for one hour. The reaction mixture was then cooled and was pipetted carefully onto a mixture of ice and 2M hydrochloric acid. The resulting mixture was extracted with ethyl acetate (3×200 mL) and the combined organic layers were washed with brine, dried over magnesium sulfate and concentrated in vacuo. The residue was purified by column chromatography on silica gel, eluting with pentane:ethyl acetate, 84:16, 80:20, 75:25 to afford the title compound as a yellow solid in 66% yield, 5.31 g.

LRMS (APCI⁺): m/z [M+H]⁺ 167

### Preparation 71

### 3,6-Dimethyl-1H-indazol-4-ol

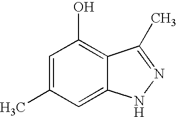

A solution of the compound of Preparation 70 (4.15 g, 25 mmol) in ethylene glycol (70 mL) was added dropwise to a solution of hydrazine monohydrate (2.4 mL, 50 mmol) in ethylene glycol (15 mL) and the mixture was stirred at room temperature for 1 hour and at 150° C. for 80 minutes. The cooled reaction mixture was then poured onto water and was acidified to pH6 with acetic acid. The aqueous mixture was extracted with ethyl acetate (4×200 mL) and the combined extracts were washed with 5% sodium sulphite (200 mL) and brine (200 mL). The organic phase was dried over magnesium sulfate and concentrated in vacuo. Purification of the residue by column chromatography on silica gel, eluting with ethyl acetate:pentane, 50:50, afforded the title compound as a yellow solid in 95% yield, 3.84 g.

LRMS (APCI⁺): m/z [M+H]⁺ 163

### Preparation 72

### 4-(tert-Butyl-dimethyl-silanyloxy)-3,6-dimethyl-1H-indazole

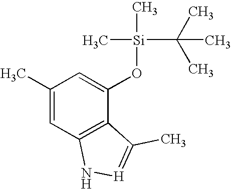

tert-Butyldimethylchlorosilane (511 mg, 3.39 mmol) and imidazole (1.05 g, 15.4 mmol) were added to a solution of the compound of Preparation 71 (500 mg, 3.08 mmol) in N,N-dimethylformamide and the reaction mixture was stirred at 0° C. for 1 hour and at room temperature for 18 hours. Additional tert-butyldimethylchlorosilane (511 mg, 3.39 mmol) was added and the reaction mixture was heated at 80° C. for 48 hours. The solvent was then evaporated under reduced pressure and the residue was partitioned between ethyl acetate and water. The organic phase was separated, washed with brine (2×10 mL), dried with magnesium sulfate and concentrated in vacuo. The residue was purified by column chromatography on silica gel, eluting with pentane:ethyl acetate, 15:85, to afford the title compound as an orange oil in 23% yield.

LRMS (APCI⁺): m/z [M+H]⁺ 27

### Preparation 73

### 4-(tert-Butyl-dimethyl-silanyloxy)-3,6-dimethyl-indazole-1-carboxylic acid tert-butyl ester

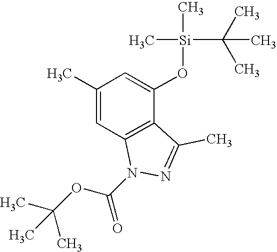

Di-tert-butyl dicarbonate (503 mg, 2.3 mmol) and 4-dimethylaminopyridine (51 mg, 0.4 mmol) were added to the compound of Preparation 72 (580 mg, 2 mmol) in dichloromethane (5 mL) and the reaction mixture was stirred for 18 hours. Water was then added to the reaction mixture and the aqueous mixture was extracted with dichloromethane (3×10 mL). The combined organic extracts was dried over sodium sulfate and concentrated in vacuo. Purification of the residue by column chromatography on silica gel, eluting with pentane:ethyl acetate, 95:5, afforded the title compound as a colourless oil in 66% yield, 527 mg.

LRMS (APCI⁺): m/z [M+H]⁺ 377

### Preparation 74

### 4-Hydroxy-3,6-dimethyl-indazole-1-carboxylic acid tert-butyl ester

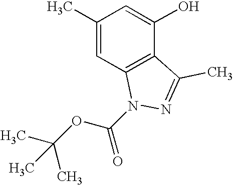

Tetrabutylammonium fluoride solution (1M in tetrahydrofuran, 21 mL, 2.1 mmol) was added dropwise to a solution of the compound of Preparation 73 (527 mg, 1.4 mmol) in tetrahydrofuran (9 mL) and the solution was stirred at 0° C. for 30 minutes and at room temperature for 5 minutes. Water was then added to the mixture and the aqueous mixture was extracted with ethyl acetate (2×10 mL). The combined organic extracts were washed with brine, dried over magnesium sulfate and concentrated in vacuo. Purification by column chromatography on silica gel, eluting with pentane:ethyl acetate, 70:30 to 60:40, afforded the title compound as a white solid in 76% yield, 280 mg.

LRMS (APCI⁻): m/z [M−H]⁻ 261

### Preparation 75

### (2R)-1-[(2R)-4-Benzoyl-2-methyl-piperazin-1-yl]-2-benzyloxy-propan-1-one

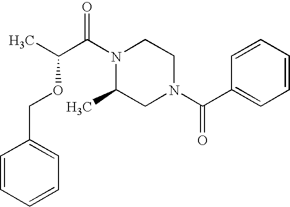

1-Hydroxybenzotriazole hydrate (825 mg, 5.3 mmol), N-methylmorpholine (808 μL, 7.3 mmol), 1-(3-dimethylaminopropyl)-3-ethylcarbodiimide hydrochloride (1.13 g, 5.8 mmol) and (3R)-(3-methyl-piperazin-1-yl)-phenyl-methanone [(1.0 g, 4.9 mmol), J. Med. Chem. 43(23), 4499; 2000] were added to a solution of (R)-(+)-2-benzyloxypropionic acid (972 mg, 5.3 mmol) in dichloromethane (10 mL) and the mixture was stirred for 18 hours. Additional (R)-(+)-2-benzyloxypropionic acid (486 mg, 2.65 mmol), 1-(3-dimethylaminopropyl)-3-ethylcarbodiimide hydrochloride (5.65 mg, 2.9 mmol), 1-hydroxybenzotriazole hydrate (413 mg, 2.65 mmol) and N-methylmorpholine (808 μL, 7.3 mmol) were added and the mixture was heated under reflux for 3 hours. Water was then added to the reaction mixture and the aqueous solution was extracted with dichloromethane (2×10 mL). The combined organic extracts were washed with 2M hydrochloric acid, 1M sodium hydroxide solution and brine. The organic phase was dried over magnesium sulfate and concentrated in vacuo. Purification by column chromatography on silica gel, eluting with pentane:ethyl acetate, 50:50, followed by ethyl acetate:methanol, 90:10, afforded the title compound as a white foam in 81% yield, 1.45 g

LRMS (APCI⁻): m/z [M−H]⁻ 367

### Preparation 76

### (2R)-1-[(2R)-4-Benzoyl-2-methyl-piperazin-1-yl)-2-hydroxy-propan-1-one

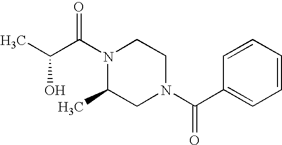

Pd(OH)₂ (300 mg, 2.2 mmol) and ammonium formate (1.37 g, 22 mmol) were added to a solution of the compound of Preparation 75 (1.45 g, 3.9 mmol) in ethanol (30 mL) and the mixture was heated at 60° C. for 2 hours. Tlc analysis then showed that the reaction had not reached completion so additional Pd(OH)₂ (300 mg, 2.2 mmol) was added, followed by ammonium formate (1.37 g, 22 mmol) added at 45 minute intervals until all of the starting material was consumed. The reaction mixture was then cooled and filtered through Arbocel®, washing through with ethanol (10 mL). The filtrate was concentrated in vacuo and the residue was purified by column chromatography on silica gel, eluting with dichloromethane:methanol, 95:5, to afford the title compound as a white gum in 91% yield, 1 g.

LRMS (APCI⁺): m/z [M+H]⁺ 277

### Preparation 77

### 4-[(1S)-2-[(2R)-4-Benzoyl-2-methyl-piperazin-1-yl]-1-methyl-2-oxo-ethoxy]-3,6-dimethyl-indazole-1-carboxylic acid tert-butyl ester

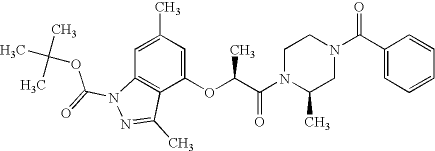

The compound of Preparation 76 (70 mg, 0.25 mmol), di-tert-butyl azodicarboxylate (233 mg, 1.01 mmol) and polymer supported triphenyl phosphine (380 mg, 1.13 mmol) were added to a solution of the compound of Preparation 74 (70 mg, 0.25 mmol) in dichloromethane (3 mL) and the reaction mixture was stirred 0° C. for 30 minutes and at room temperature for 18 hours. The reaction mixture was then filtered through a filter tube, washing through with dichloromethane. The filtrate was washed with sodium hydroxide solution, dried over magnesium sulfate and concentrated in vacuo. Purification of the residue by column chromatography on silica gel, eluting with pentane:ethyl acetate, 40:60 to 20:80 afforded the title compound as a white foam in 34% yield, 45 mg

LRMS (APCI⁺): m/z [M+H]⁺ 521

Biological Data

The ability of the compounds of formula (I) and their pharmaceutically acceptable salts, solvates and derivatives to modulate gp120 activity, in particular inhibit the interaction of gp120 with CD4, is demonstrated using a gp160 induced cell-cell fusion assay to determine the IC₅₀ values of compounds against HIV-1 fusion. The gp160 induced cell-cell fusion assay uses a HeLa P4 cell line and a CHO-Tat10 cell line.

The HeLa P4 cell line expresses CCR5 and CD4 and has been transfected with HIV-1 LTR-β-Galactosidase. The media for this cell line is Dulbecco modified eagle's medium (D-MEM) (without L-glutamine) containing 10% foetal calf serum (FCS), 2 mM L-glutamine penicillin/streptomycin (Pen/Strep; 100 U/mL penicillin+10 mg/mL streptomycin), and 1 μg/ml puromycin.

The CHO cell line is a Tat (transcriptional trans activator)-expressing clone from a CHO JRR17.1 cell line that has been transfected with pTat puro plasmid. The media for this cell line is rich medium for mammalian cell culture originally developed at Roswell Park Memorial Institute RPMI1640 (without L-glutamine) containing 10% FCS, 2 mM L-glutamine, 0.5 mg/ml Hygromycin B and 12 μg/ml puromycin. The CHO JRR17.1 line expresses gp160 (JRFL) and is a clone that has been selected for its ability to fuse with a CCR5/CD4 expressing cell line.

Upon cell fusion, Tat present in the CHO cell is able to transactivate the HIV-1 long terminal repeat (LTR) present in the HeLa cell leading to the expression of the β-Galactosidase enzyme. This expression is then measured using a Fluor Ace™ β-Galactosidase reporter assay kit (Bio-Rad cat no. 170-3150). This kit is a quantitative fluorescent assay that determines the level of expression of β-galactosidase using 4-methylumbelliferul-galactopyranoside (MUG) as substrate. β-Galactosidase hydrolyses the fluorogenic substrate resulting in release of the fluorescent molecule 4-methylumbelliferone (4MU). Fluorescence of 4-methylumbelliferone is then measured on a fluorometer using an excitation wavelength of 360 nm and emission wavelength of 460 nm.

Compounds that inhibit fusion will give rise to a reduced signal and, following solubilisation in an appropriate solvent and dilution in culture medium, a dose-response curve for each compound can be used to calculate IC₅₀ values.

All the Examples of the invention have IC₅₀ values, according to the above method, of less than 1.5 μM. IC₅₀ values for the compounds of Examples 12, 29 and 44 are, respectively, 15 nM, 134 nM and 825 nM.

The ability of compounds of formula (I) to inhibit the interaction of gp120 with CD4 is further demonstrated using an enzyme linked immunosorbent assay (ELISA). Maxisorp plates (Nunc) are coated with 2 μg/well of anti-gp120 antibody (D7324). 100 μl of gp120 (dilution pre-determined by titration) is added to each well and incubated for 90 minutes at room temperature. The sample is removed and the wells are washed with PBS (phosphate buffered saline)+0.01% TWEEN® (polyethylene glycol sorbitan monolaurate). 50 μl/well of compound is added followed by 50 μl (0.1 μg) of soluble CD4 conjugated to horseradish peroxidase (Autogen Bioclear). The plate is incubated for 90 minutes at room temperature before the wells are washed again. The substrate OPD (o-phenylenediamine, Sigma) is added at a concentration of 0.5 mg/ml and the plate incubated in the dark at room temperature for 3 minutes before 3M HCl is added to stop the reaction. Compounds that inhibit the interaction of gp120 with soluble CD4 will give rise to a reduced absorbance at 492 nm.

All the Examples of the invention have IC₅₀ values, according to the above method, of less than 15 μM. IC₅₀ values for the compounds of Examples 1, 11 and 12 are, respectively, 0.92 μM, 1.0 μM and 0.75 μM.

1. A compound of formula (I) 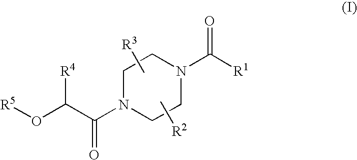
or a pharmaceutically acceptable salt, solvate or derivative thereof, wherein:
R¹ is phenyl or pyridyl, wherein said phenyl or pyridyl is optionally substituted by 1 or 2 atoms or groups selected from halo, C₁-C₆ alkoxy, CF₃, OCF₃, or CN;
R² and R³ are independently H, or C₁-C₆ alkyl;
R⁴ is C₁-C₆ alkyl;
R⁵ is phenyl; naphthyl; or a C-linked, 6 to 10 membered, mono- or bicyclic, aromatic or partially saturated, heterocycle wherein said heterocycle contains 1 to 4 nitrogen heteroatom(s), 1 or 2 nitrogen and 1 oxygen heteroatoms, or 1 or 2 nitrogen and 1 sulphur heteroatoms; wherein said phenyl, napthyl or heterocycle is optionally substituted by 1 to 3 atoms or groups selected from C₁-C₆ alkyl, C₁-C₆ fluoroalkyl, C₃-C₇ cycloalkyl, phenyl, OH, C1-C₆ alkoxy, C₁-C₆ alkoxy C₁-C₆ alkyl, OC₁-C₆fluoroalkyl, C₀-C₂ alkylene NR⁶R⁷, halo C₀-C₂ alkylene CN, C₀-C₂ alkylene CO₂R⁸, C₀-C₂ alkylene CONR⁶R⁷, C₀-C₂ alkyl SR⁹, C₀-C₂ alkylene SOR⁹, C₀-C₂ alkylene SO₂R⁹, C₀-C₂ alkylene SO₂NR⁶R⁷, C₀-C₂ alkylene NR⁸COR⁹, C₀-C₂ alkylene NR⁸CONR⁶R⁷, C₀-C₂ alkylene NR⁸SO₂R⁹, or C₀-C₂ alkylene R¹⁰, or, where R⁵ is a heterocycle, oxo;
R⁶ and R⁷ are independently H, C₁-C₆ alkyl, C₃-C₇ cycloalkyl, phenyl or R¹⁰; or when taken together with the nitrogen to which they are attached form an optionally substituted azetidine, pyrrolidine, piperidine, morpholine, or thiomorpholine ring; wherein said substituents are 1 or 2 groups selected from C₁-C₆ alkyl, or C₀-C₆ alkylene NH₂;
R⁸ is H, C₁-C₆ alkyl or phenyl;
R⁹ is C₁-C₆ alkyl or phenyl; and
R¹⁰ is imidazolyl, triazolyl, thienyl, furyl, thiazolyl, oxazolyl, thiadiazolyl, oxadiazolyl, pyridinyl, pyrimidinyl, pyridazinyl, pyrazinyl, quinolinyl, isoquinolinyl, benzimidazolyl, quinazolinyl, phthalazinyl, benzoxazolyl or quinoxalinyl, each optionally substituted by 1 to 3 atoms or groups selected from C₁-C₆ alkyl, C₁-C₆ alkoxy, cyano or halo.

2. A compound according to claim 1 wherein R¹ is phenyl or pyridyl, wherein said phenyl or pyridyl is optionally substituted by 1 or 2 atoms or groups selected from halo.

3. A compound according to claim 1 wherein R¹ is phenyl, fluorophenyl or pyridyl.

4. A compound according to any one of claims 1 to 3 wherein R¹ is phenyl.

5. A compound according to any one of claims 1 to 4 wherein R² is C₁-C₄alkyl.

6. A compound according to any one of claims 1 to 5 wherein R² is methyl.

7. A compound according to any one of claims 1 to 6 wherein R³ is H.

8. A compound according to any one of claims 1 to 7 wherein R⁴ is C₁-C₄alkyl.

9. A compound according to any one of claims 1 to 8 wherein R⁴ is methyl.

10. A compound according to any one of claims 1 to 9 wherein R⁵ is an optionally substituted phenyl, naphthyl, pyridyl, indazolyl, benzimidazolyl, quinolinyl, isoquinolinyl, quinazolinyl, benzopiperidinyl or benzoxazolyl; wherein said substituents are 1 to 3 atoms or groups selected from C₁-C₆ alkyl, C₁-C₆ alkoxy, halo, CN, CO₂R⁸, CONR⁶R⁷, or R¹⁰.

11. A compound according to any one of claims 1 to 10 wherein R⁵ is an optionally substituted phenyl or pyridyl, wherein said substituents are 1 to 3 groups selected from C₁-C₆ alkoxy, CO₂R⁸, or CONR⁶R⁷.

12. A compound according to any one of claims 1 to 11 wherein R⁶ is H or C₁-C₄ alkyl.

13. A compound according to any one of claims 1 to 12 wherein R⁷ is H, C₁-C₄ alkyl or C₃-C₆ cycloalkyl.

14. A compound according to any one of claims 1 to 13 wherein R⁸ is C₁-C₄ alkyl.

15. A compound according to any one of claims 1 to 14 wherein R¹⁰ is imidazolyl, pyrazolyl, triazolyl or oxadiazolyl, each optionally substituted by 1 to 3 atoms or groups selected from C₁-C₄ alkyl, C₁-C₄ alkoxy, cyano or halo.

16. A compound of formula (Ia) 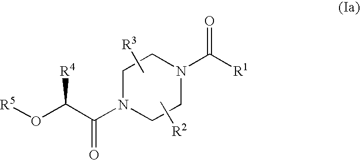
or a pharmaceutically acceptable salt, solvate or derivative thereof, wherein R¹, R², R³, R⁴ and R⁵ are as defined in any one of claims 1 to 15.

17. A compound of formula (Ib) 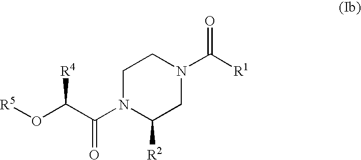
or a pharmaceutically acceptable salt, solvate or derivative thereof, wherein R¹, R², R⁴ and R⁵ are as defined in any one of claims 1 to 15.

18. A compound selected from the group consisting of:
(2S)-1-[(2R)-4-Benzoyl-2-methyl-piperazin-1-yl)]-2-(3-methyl-1H-indazol-4-yloxy)-propan-1-one;
(2S)-1-[(2R)-4-Benzoyl-2-methyl-piperazin-1-yl]-2-[2-(2H-pyrazol-3-ylamino)-quinolin-5-yloxy]-propan-1-one;
5-{(1S)-2-[(2R)-4-Benzoyl-2-methyl-piperazin-1-yl]-1-methyl-2-oxo-ethoxy}-isoquinoline-1-carboxylic acid methylamide;
(2S)-1-[(2R)-4-Benzoyl-2-methyl-piperazin-1-yl]-2-(8-chloro-2-methylamino-quinolin-5-yloxy)-propan-1-one;
(2S)-1-[(2R)-4-Benzoyl-2-methyl-piperazin-1-yl]-2-[1-(2H-pyrazol-3-ylamino)-isoquinolin-5-yloxy]-propan-1-one;
4-{(1S)-2-[(2R)-4-Benzoyl-2-methyl-piperazin-1-yl]-1-methyl-2-oxo-ethoxy}-3-methoxy-N-methyl-benzamide;
5-{(1S)-2-[(2R)-4-Benzoyl-2-methyl-piperazin-1-yl]-1-methyl-2-oxo-ethoxy}-methoxy-pyridine-2-carboxylic acid methylamide;
5-{(1S)-2-[(2R)-4-Benzoyl-2-methyl-piperazin-1-yl]-1-methyl-2-oxo-ethoxy}-4-methoxy-pyridine-2-carboxylic acid amide;
5-{(1S)-2-[(2R)-4-Benzoyl-2-methyl-piperazin-1-yl]-1-methyl-2-oxo-ethoxy}-4-methoxy-pyridine-2-carboxylic acid ethylamide;
5-{(1S)-2-[(2R)-4-Benzoyl-2-methyl-piperazin-1-yl]-1-methyl-2-oxo-ethoxy}-4-methoxy-pyridine-2-carboxylic acid cyclopropylamide;
and the pharmaceutically acceptable salts, solvates or derivatives thereof.

19. A pharmaceutical composition comprising a compound of formula (I) or a pharmaceutically acceptable salt, solvate or derivative thereof, according to any one of claims 1 to 18, together with one or more pharmaceutically acceptable excipients, diluents or carriers.

20. A pharmaceutical composition according to claim 19 further comprising one or more additional therapeutic agents.

21. A compound of formula (I) or a pharmaceutically acceptable salt, solvate or derivative thereof according to any of claims 1 to 18 for use as a medicament.

22. A compound of formula (I) or a pharmaceutically acceptable salt, solvate or derivative thereof according to any of claims 1 to 18 for use in the treatment of a HIV, a retroviral infection genetically related to HIV, or AIDS.

23. The use of a compound of formula (I) or a pharmaceutically acceptable salt, solvate or derivative thereof as claimed in any one of claims 1 to 18 for the manufacture of a medicament for the treatment of a HIV, a retroviral infection genetically related to HIV, or AIDS.

24. A method of treatment of a mammal suffering from HIV, a retroviral infection genetically related to HIV, or AIDS, said method comprising administering to said mammal an effective amount of a compound of formula (I) or a pharmaceutically acceptable salt, solvate or derivative thereof according to any one of claims 1 to 18.

25. A compound of formula (II), (IV) or (VII) 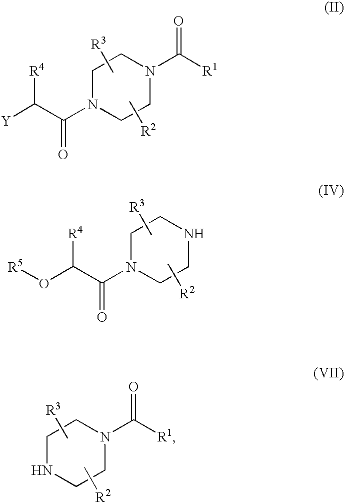
wherein:
R¹ is phenyl or pyridyl, wherein said phenyl or pyridyl is optionally substituted by 1 or 2 atoms or groups selected from halo, C₁-C₆ alkoxy, CF₃, OCF₃, or CN;
R² and R³ are independently H, or C₁-C₆alkyl;
R⁴ is C₁-C₆ alkyl; and
R⁵ is phenyl; naphthyl; or a C-linked, 6 to 10 membered, mono- or bicyclic, aromatic or partially saturated, heterocycle wherein said heterocycle contains 1 to 4 nitrogen heteroatom(s), 1 or 2 nitrogen and 1 oxygen heteroatoms, or 1 or 2 nitrogen and 1 sulphur heteroatoms; wherein said phenyl, napthyl or heterocycle is optionally substituted by 1 to 3 atoms or groups selected from C1-C6 alkyl, C1-C6 fluoroalkyl, C3-C7 cycloalkyl, phenyl, OH, C1-C6 alkoxy, C1-C6 alkoxy C1-C6 alkyl, OC1-C6fluoroalkyl, C0-C2 alkylene NR6R7, halo, C0-C2 alkylene CN, C0-C2 alkylene CO2R8, C0-C2 alkylene CONR6R7, C0-C2 alkylene SR9, C0-C2 alkylene SOR9, C0-C2 alkylene SO2R9, C0-C2 alkylene SO2NR6R7, C0-C2 alkylene NR8COR9, C0-C2 alkylene NR8CONR6R7, C0-C2 alkylene NR8SO2R9, or C0-C2 alkylene R10, or, where R5 is a heterocycle, oxo.
